(19) United States
(12) Patent Application Publication
(10) Pub. No.: US 2022/0202868 A1
(43) Pub. Date: Jun. 30, 2022

(54) IMPROVED STEM CELL POPULATIONS FOR ALLOGENEIC THERAPY

(71) Applicant: TiGenix S.A.U., Madrid (ES)

(72) Inventors: Olga DE LA ROSA MORALES, Madrid (ES); Alvaro AVIVAR-VALDERAS, Madrid (ES)

(73) Assignee: TiGenix S.A.U., Madrid (ES)

(21) Appl. No.: 17/434,327

(22) PCT Filed: Feb. 26, 2020
(86) PCT No.: PCT/EP2020/055017

§ 371 Date: Aug. 26, 2021

(30) Foreign Application Priority Data
Feb. 27, 2019 (EP) 19382141.0

(51) Int. Cl. A61K 35/28; C12N 5/0775; G01N 33/50; C07K 16/28

(52) U.S. Cl. CPC A61K 35/28; C12N 5/0667; C07K 16/2833; G01N 33/5073; C12N 5/0663

(57) ABSTRACT

The present invention relates to an improved stem cell population for allogeneic stem cell therapy, in particular for the treatment of presensitized patients and for retreatment. Further, methods for obtaining said stem cell populations are provided. In addition, the present invention relates to pharmaceutical compositions comprising said stem cell populations and their use in allogeneic stem cell therapy.

### TECHNICAL FIELD

The present invention relates to an improved stem cell population for allogeneic stem cell therapy, in particular for the treatment of presensitized patients and for retreatment. Further, methods for obtaining said stem cell populations are provided. In addition, the present invention relates to pharmaceutical compositions comprising said stem cell populations and their use in allogeneic stem cell therapy.

### BACKGROUND OF THE INVENTION

Stem cells for use in research and medical applications may be derived from embryonal, fetal or adult tissue and include embryonic stem cells (ESCs), umbilical cord stem cells, induced pluripotent stem cells (iPSCs) and adult stem cells from different sources. Numerous clinical trials are currently under way or have been successfully concluded for the treatment of fistulas, leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease. An overview of current medical applications for the different types of stem cells may be found in Mahla RS, International Journal of Cell Biology. 2016 (7): 1-24.

Mesenchymal stem cells (MSCs) are multipotent stromal cells that can differentiate into a variety of cell types including osteoblasts, chondrocytes, myocytes and adipocytes.

The use of autologous and allogeneic MSCs for treating a variety of conditions has been evaluated across several clinical trials (Galipeau and Sensebe, (2018), Cell Stem Cell 22, 824-833, in particular Table S1) including those focused on the treatment of damaged gastrointestinal tracts, sepsis and graft versus host disease as well as several autoimmune and inflammatory diseases.

Adult MSCs can be found in nearly all tissues and are predominantly located in the perivascular niches. Although the vast majority of clinical trials have been carried out using bone marrow MSC (BM-MSC), there is an increasing number of trials using MSCs from other sources, such as adipose tissue. BM-MSCs and adipose-derived mesenchymal stem cells (ASCs) share similar potency capabilities and replicative ratios. ASCs, however, confer advantages of being more easily harvested and abundant in samples for culturing (100 times more abundant in one gram of fat tissue compared with BM tissue).

Standard immunologic dogma holds that any foreign tissue will elicit an immune reaction in an organism. This concept is clearly evident in solid organ and hematopoietic transplantation, in which aggressive immunosuppression is the norm to protect allografts from rejection. The strongest evidence for a role for antibodies in graft rejection is the hyperacute rejection of primarily vascularized organs, such as the kidney and heart. High titers of antidonor antibodies can be demonstrated in recipients presenting with these reactions. These antibodies combine with HLA antigens on endothelial cells, with subsequent complement fixation and accumulation of polymorphonuclear cells. Endothelial damage then occurs probably as a result of enzymes released from polymorphonuclear leukocytes; platelets then accumulate, thrombi develop, and the result is renal cortical necrosis or myocardial infarction. In order to avoid this anti-HLA-antibodies mediated reaction, HLA typing is performed in clinical organ and tissue transplantation in order to match the donor and the recipient as closely as possible to avoid transplant rejection.

As the field of cell-based therapy evolves, it has become evident that various cell types—mesenchymal stem cells (MSCs) being the prototype—may have the ability to evade and/or suppress the immune system so that in some studies the MSCs have been applied without concomitant immunosuppression.

Griffin et al., Immunol. & Cell Biol. (2012), 1-12 have reviewed experimental data to determine whether good in vivo evidence exists in support of the “immune privileged” status of allo-MSCs. They considered published studies regarding the immunogenicity of allo-MSCs following activation by inflammatory stimuli or following differentiation.

The authors summarize that the majority of these studies has documented specific cellular (T-cell) and humoral (B-cell/antibody) immune responses against donor antigens following administration of non-manipulated, IFN-γ activated and differentiated allo-MSCs with accelerated rejection of subsequent allogeneic transplants to apparent promotion of donor-specific tolerance. In some of these studies an accelerated rejection of the transplant or the administered MSCs (see Tables 1 and 2 of Griffin et al.) was observed. The authors recommend that the concept of the immune privileged nature of allo-MSCs be reconsidered and the range and clinical implications of anti-donor immune responses elicited by allo-MSCs be more precisely studied. Consequently, the anti-donor immune response may have implications for the efficacy due to improved clearance of allo-MSCs and/or adverse effects in allogeneic therapy of humans and animals.

In view thereof, there is a need for means suitable for allogeneic therapy of patients, wherein the means exhibit a reduced immunogenicity and/or increased suitability in allogeneic therapy with presensitized patients and/or for retreatment. Further, methods for obtaining them as well as medical uses of them shall be provided as well.

### SUMMARY OF THE INVENTION

The technical problems underlying the invention are solved by the provision of the subject-matter as defined in the claims.

According to a first aspect is provided an in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample);
b) contacting the test sample and the control sample with a range of different concentrations of an HLA-Class I antibody under conditions such that the HLA-Class I antibody binds to HLA-Class I expressed in the test sample and the control sample;
c) adding complement to the test sample and the control sample such that the bound HLA-Class I antibody is saturated with complement and complement-dependent cytotoxicity (CDC) is induced;
d) determining the CDC for the range of different concentrations of the HLA-Class I antibody by measuring the cell lysis induced in the test sample and the control sample;
e) determining the concentration of HLA-Class I antibody in the test sample and the control sample that induces 50% of the maximal CDC (EC₅₀ value); and
f1) selecting the SC population for allogeneic therapy if the ratio of the EC₅₀ value of the control sample to the EC₅₀ value of the test sample is less than 1.25, preferably less than 1.0, more preferably less than 0.5; particularly preferably less than 0.25; or
f2) selecting the SC population for allogeneic therapy if the EC₅₀ value of the test sample is at least 3.5 ng/ml, preferably at least 9 ng/ml, more preferably at least 15 ng/ml, particularly preferably at least 20 ng/ml of the HLA-Class I antibody.

According to a second aspect is provided an SC population suitable for allogeneic therapy, in particular for the treatment of presensitized patients or retreatment with allogeneic therapy, having any of the following properties:
i) a ratio of the EC₅₀ value of the control sample to the EC₅₀ value of the test sample is less than 1.25, preferably less than 1.0, more preferably less than 0.5 and particularly preferably less than 0.25, wherein the EC₅₀ value is determined as set forth in the method according to the invention;
ii) an EC₅₀ value of the test sample of at least 3.5 ng/ml HLA-Class I antibody, preferably at least 9 ng/ml, more preferably at least 15 ng/ml, particularly preferably at least 20 ng/ml HLA-Class I antibody, wherein the EC₅₀ value is determined as set forth in the method according to the invention; and/or
iii) a ratio of CD46 expression in the test sample to the CD46 expression in the control sample is more than 2.0, preferably more than 2.5, particularly preferably more than 3.0, wherein the CD46 expression is determined as set forth in the method of the invention.

According to a third aspect is provided a pharmaceutical composition comprising the SC population according to the invention and optionally a pharmaceutically acceptable carrier.

According to a fourth aspect is provided a method for preparing a pharmaceutical composition comprising the steps:
a) performing the method according to the invention;
b) formulating the selected SC population with at least one pharmaceutically acceptable carrier.

According to a fifth aspect is provided the use of the SC population according to the invention in allogeneic stem cell therapy in a patient in need thereof, preferably to a presensitized patient or a patient undergoing retreatment.

According to a sixth aspect is provided an allogeneic stem cell therapy method comprising administering the SC population according to the invention to a patient in need thereof, preferably in a presensitized patient or a patient undergoing retreatment.

The inventors have investigated whether allo-sensitization takes place when an ASC population is administered to patients suffering from Crohn's disease and treatment-refractory, draining complex perianal fistulas, wherein the ASC population is administered intra-lesionally to allogeneic, non-HLA-matched recipients. It was observed that while at time 0 only 4 patients showed an antibody reactivity against HLA-Class I molecules, 22 patients showed this antibody reactivity after 12 weeks of treatment. In the control group only an increase of reactivity from 6 patients (at time 0) to 9 patients after 12 weeks was observed. Subsequently, ASC cell populations from different donors were investigated. It was found that plasma samples from presensitized patients and patients developing an anti-HLA class I reactivity differed significantly in their binding behaviour and their ability to induce complement-dependent cytotoxicity (CDC) in IFN-γ induced ASC populations from different donors. Based on these findings, the inventors further determined that SC populations from different donors differ significantly in their HLA-Class I binding and in their sensitivity to cell lysis due to activation of the classical complement pathway (antibody/complement complex formation), wherein SC populations being more resistant to alloreactivity in vitro are preferable for purposes of allogeneic therapy. This applies in particular to situations where the patients are presensitized to HLA-Class I antigens e.g. by earlier pregnancies, or in cases of retreatment where the same donor SC population is administered multiple times to the same patient. The inventors have further determined that the increased resistance to alloreactivity is mediated by CD46 expression and that inhibition of CD46 expression in ASC cells correlates with enhanced CDC sensitivity.

### BRIEF DESCRIPTION OF THE DRAWINGS

FIG. 1 shows the Characterization of DSA generation in ADMIRE CD1. A. Distribution of Labscreen Mixed/Labscreen Single Antigen (LSM/LSA) (Tait et al. (2013), Transplantation 95: 19-47) results for indicated visits: pre-treatment (baseline), W12 and W52 in both placebo (lower chart) and Cx601 (upper chart) arms of the study. A total of five (Cx601 arm) and 12 (placebo arm) patients withdrew from the study and, therefore, no LSM/LSA data was available. B. Kinetic curves illustrating anti-HLA Abs titer represented as the sum of mean fluorescence intensity (ΣMFI) from each micro-sphere measured with the LSM assay at the indicated time points. The dotted line in each graph indicates the MFI >3,000 threshold used for determining positivity. Circle in upper right panel, indicates patient 92 (Pat92). C. Graph representing HLA incompatibility between each patient and ASCs. For the correlation of incompatibility the percentage of individuals that generated DSAs was plotted versus the number (range) of mismatched eplets (unshared, unique chain of polymorphic residues found in a locus) (Duquesnoy (2002) Hum. Immunol. 63: 339-352). For linear regression the Pearson test (r²) was applied. P values were determined by the Student's t-test.

FIG. 2 shows the HLA expression in ASCs and in vitro anti-HLA Ab binding. A. Graphs showing the correlation between MFI increase and each concentration of class I HLA (W6/32) Ab and class II HLA (L243) Ab directed against untreated (black circle) and pre-activated with IFNγ (white square) ASCs. B. Plots of FcTox (complement-dependent cytotoxicity by flow cytometry assay) representing a negative control (isotype), a positive control (hyper-immunized samples, HI pool) and patient 92 (Pat92, naïve patient that generated de novo DSA) serum. Lower left panel shows FACS binding strength quantification (total number of IgG+ cells) using isotype control (light grey), hyper-immunized serum (dark grey) or Pat92 serum (mid-dark grey). Lower right panel shows FACS cell death quantification (total number of 7-AAD+ cells) in control conditions (no rabbit complement, light grey) hyper-immunized serum or Pat92 serum (mid-dark grey). P values were determined by the Student's t-test and r² by Pearson test.

FIG. 3 shows that ADMIRE CD1 plasma samples induce low cytotoxic killing in ASCs in vitro. A. Graphs showing normalized percent values of HLA-I binding in 10 pre-sensitized (upper panels) and 17 de novo DSA+ patients (lower panels) at the indicated time points (W0 pre-treatment and W12 post-treatment). Prior to binding assay DonA (donor administered in the ADMIRE CD1 trial) and DonB, ASCs were grown under normal (basal) conditions or in the presence of 3 ng/mL IFNγ for 48 hours. B. Graphs showing normalized percent values of 7-AAD positive ASC in 10 pre-sensitized (upper panels) and 17 de novo DSA+patients (lower panels) at the indicated time points. P values were determined by the Student's T-test.

FIG. 4 shows that ASCs express high levels of mCRP. A. Graphs showing MFI values of CD46, CD55 and CD59 in seven ASC donors (black bars) and one BM-MSC donor (white bars) via FACS analysis. Cells were grown in the presence of 3 ng/mL (IFNγ) for 48 hours or left untreated (basal). B. Graphs showing differential MFI values of CD46, CD55 and CD59 in the seven ASC donors determined by FACS analysis. Cells were grown in the presence of 3 ng/mL IFNγ for 48 hours (IFNγ) or left untreated (basal). C. Graphs showing antibody binding (upper panel) and cell death (lower panel) dependent on the antibody concentration for ASCs derived from donors DonA, DonB, DonC, DonD, DonE, DonF and DonG cultured under basal conditions (left-hand panels) and with IFNγ (right-hand panels). P-values were determined by the Student's t-test. From the lower right panel the EC₅₀ values can be determined.

FIG. 5 shows that CD46 mediates complement cytotoxicity in ASCs. A. Graph showing percentage of 7-AAD positive parental and CD46^{KO} DonB ASCs against increased concentration levels of W6/32 Ab. Prior to analysis parental and CD46^{KO} DonB ASCs were grown in the presence of 3 ng/mL IFNγ for 48 hours (IFNγ) or left untreated (basal). B. Sigmoidal curves displaying the percentage of 7-AAD positive parental and CD46^{KO} ASCs against the concentration of W6/32 Ab (transformed from linear to log₁₀). P values were determined from the two way ANOVA test.

FIG. 6A-B relates to a correlation of MFI values of W6/32 (A) and CD46, CD55 and CD59 (B) of ASCs donor grown in the presence of 3 ng/mL IFNγ for 48 hours (white squares) or basal conditions (black circles). P values show the slope significance from the linear regression. The R-squared value for the significance of the slopes in IFNγ conditions for CD46 and CD55 (B) was 0.74 and 0.71, respectively. C. Graph showing CD46 MFI values in parental (white bar) and CD46^{KO} (black and grey bars) ASCs. Prior to analysis parental and CD46^{KO} ASCs were grown with 3 ng/mL IFNγ for 48 hours (IFNγ) or were untreated (basal). P values were determined by the Student's t-test.

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

According to a first aspect is provided an in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample);
b) contacting the test sample and the control sample with a range of different concentrations of an HLA-Class I antibody under conditions such that the HLA-Class I antibody binds to HLA-Class I expressed in the test sample and the control sample;
c) adding complement to the test sample and the control sample such that the bound HLA-Class I antibody is saturated with complement and complement-dependent cytotoxicity (CDC) is induced;
d) determining the CDC for the range of different concentrations of the HLA-Class I antibody by measuring the cell lysis induced in the test sample and the control sample;
e) determining the concentration of HLA-Class I antibody in the test sample and the control sample that induces 50% of the maximal CDC (EC₅₀ value); and
f1) selecting the SC population for allogeneic therapy if the ratio of the EC₅₀ value of the control sample to the EC₅₀ value of the test sample is less than 1.25, preferably less than 1.0, more preferred less than 0.5; particularly preferred less than 0.25; or
f2) selecting the SC population for allogeneic therapy if the EC₅₀ value of the test sample is at least 3.5 ng/ml, preferably at least 9 ng/ml, more preferred at least 15 ng/ml, particularly preferred at least 20 ng/ml of the HLA-Class I antibody.

“A stem cell (SC) population” herein means any stem cell including embryonic pluripotent stem cells (ESCs), fetal stem cells and adult stem cells. The population may be either a primary cell culture, a cell line or derived from a clone.

The population of stem cells may be a population of pluripotent stem cells or a population of mesenchymal stem cells (MSCs), e.g. bone-marrow derived, umbilical cord tissue-derived, blood-derived (including cord blood derived), menstrual, dental pulp-derived, placental-derived or adipose-derived MSCs (Huang et al., J Dent. Res. (2009) 88(9): 792-806; Carvalho et al., Curr. Stem Cell Res. Ther. (2011) 6(3): 221-8; Harris et al., Curr Stem Cell Res Ther. (2013) 8(5): 394-9; Li et al., Ann. N Y Acad. Sci. (2016) 1370(1): 109-18). In a preferred embodiment, the stem cells are human stem cells (e.g. human ASCs). In preferred embodiments of the invention, the population of stem cells are adipose-derived stromal stem cells (ASCs). The ASCs may be an expanded population of ASCs.

Methods for producing and culturing populations of stem cells are well known.

The population of stem cells may be substantially pure. The term “substantially pure” in relation to a population of stem cells (e.g. a MSC population such as a population of ASCs) refers to a stem cell population that is least about 75%, typically at least about 85%, more typically at least about 90%, and most typically at least about 95% homogenous. Homogeneity can be assessed by morphology and/or by cell surface marker profile. Techniques for assessing morphology and cell surface marker profile are disclosed herein.

The stem cells are characterized by their self-renewal ability in undifferentiated state and their capacity to differentiate into specialized cell types. ESCs are publicly available from clones. Adult stem cells are undifferentiated cells including hematopoietic stem cells, mammary stem cells, intestinal stem cells, mesenchymal stem cells, neural stem cells, olfactory adult stem cells, neural crest stem cells and testicular cells. Preferably, the SC population is a mesenchymal stem cell (MSC), more preferred a human MSC population.

Pluripotent Stem Cells

There are two sources of pluripotent stem cells. First, embryonic stem cells (ESCs) are derived from the inner cell mass of a pre-implantation blastocyst and pluripotency is controlled by an intrinsic regulatory network of core transcription factors, octamer-binding transcription factor 4 (OCT4), sex determining region Y-box 2 (SOX2), and Nanog homeobox (NANOG). Second, induced pluripotent stem cells (iPSCs) are derived by the ectopic or elevated expression of four transcription factors, OCT4, SOX2, Kruppel like factor 4 (KLF4), and MYC proto-oncogene MYC) essential for induction of pluripotency in somatic cells.

Techniques for isolating stable (undifferentiated) cultures of embryonic stem cells, such as human embryonic stem cells, are well established (e.g. U.S. Pat. No. 5,843,780; Thomson et al., Science (1998) 282: 1145-1147; Turksen & Troy (2006) Human Embryonic Stem Cells. In: Turksen K. (eds) Human Embryonic Stem Cell Protocols. Methods in Molecular Biology, volume 331, Humana Press; Sevilla et al., Stem Cell Research (2017) 25: 217-220; and Mitalipova & Palmarini (2006) Isolation and Characterization of Human Embryonic Stem Cells. In: Turksen K. (eds) Human Embryonic Stem Cell Protocols. Methods in Molecular Biology, volume 331, Humana Press).

Techniques for producing iPSCs are well established since their discovery in 2007 by Yamanaka's group (e.g. Takahashi et al., Cell (2007) 131(5): 861-72). Since then, new improved methods for iPSC generation have been developed, including non-integration and feeder free methodologies and automated high-throughput derivation (Paull et al., Nature Methods (2015) 12(9): 885 892).

iPSCs are characterized by the expression of a battery of pluripotency markers: NANOG, SOX2, SSEA4, TRA1-81, TRA1-60, and the lack of lineage-specific markers. The pluripotency of iPSC is demonstrated by their capacity to differentiate into the three germ layers in the embryoid body assay, with posterior analysis of differentiation markers from the three germ layers Tuj1 (ectoderm marker), SMA (mesoderm marker) and SOX17 (endoderm marker) by immunohistochemistry (Paull et al., Nature Methods (2015) 12(9): 885 892.

Induced pluripotent stem cells (iPSCs) are derived by the ectopic or elevated expression of four transcription factors, OCT4, SOX2, Kruppel like factor 4 (KLF4), and MYC proto-oncogene (C-MYC) essential for induction of pluripotency in somatic cells.

MSCs

“Mesenchymal stem cells” (also referred to herein as “MSCs”) are multipotent stromal cells. They are typical derived from connective tissue, and are non-hematopoietic cells. The population of MSCs (according to Dominici et al. (2006), Cytotherapy 8(4): 315-317), may: (1) adhere to plastic under standard culture conditions (e.g. a minimal essential medium plus 20% fetal bovine serum); (2) express (i.e. greater than or equal to 80% of population of MSCs) CD105, CD90, CD73 and CD44; (3) lack expression (e.g. less than or equal to 5% of the MSC population) of CD45, CD14 or CD11b, CD790L or CD19, and HLA-DR (HLA Class II); (4) be capable of differentiating into osteoblasts, adipocytes and chondroblasts.

MSCs can be obtained using standard methods from, for example, bone marrow, umbilical cord tissue and blood, menstrual, dental pulp, cord blood, placental and adipose tissues. Although MSCs obtained from different tissues are similar, they have some differences in phenotypical and functional characteristics. For example, the expression levels of cell surface markers CD54 and CD106 may differ depending on the source/origin of the MSCs. These can be measured by flow cytometry. The mRNA levels of some genes such as SOX2, IL1alpha, IL1 beta, IL6 and IL8, may be differentially expressed by MSCs from different tissues, and can be measured by routine methods. IL6 and PGE2 secretion may also be different between MSC from different origins, and thus the cells may have different modulatory capacity (see, e.g. Yang et al. PLoS ONE (2013) 8(3) e59354).

Bone Marrow Derived MSCs (BMSCs)

Bone-marrow mesenchymal stem cells (BM-MSCs) are similar to MSCs from other tissue sources. However, they have some differences in phenotypical and functional characteristics compared to MSCs from other tissue origins, such as umbilical-cord MSCs, placental MSCs, dental pulp MSCs, and menstrual MSCs. Even though their minimal characterization criteria is common, including their capacity to adhere to plastic, minimal surface identity markers and capacity to differentiate into bone, cartilage, tendon and fatty tissue, they all have some slight differences. These peculiarities include different expression levels of some surface markers, such as CD105, different levels of secreted soluble factors implicated in their immunomodulatory potential and regenerative potential, and in general, slightly different functional properties that may make each source or origin more suitable for specific therapeutic indications (Miura et al., Int J Hematology (2016) 103(2): 122-128; Wuchter et al., Cytotherapy (2015) 17(2): 128-139; Wright et al., Stem Cells (2011) 29(2): 169-178).

Umbilical Cord Derived and Dental Pulp Derived MSCs

Huang et al. (J. Dent. Res. (2009) 88(9): 792-806) discusses MSCs from dental pulp and compares their characteristics with MSCs from other sources. Carvalho et al. (Curr Stem Cell Res Ther. (2011) 6(3): 221-228) and Harris et al. (Curr Stem Cell Res Ther. (2013) 8(5): 394-399) discuss umbilical cord-derived MSCs, their characterisation (including phenotype and secretome) and applications thereof.

ASCS

Adipose-derived MSCs (ASCs) are normally isolated from subcutaneous adipose tissue, which allows them to be acquired in large numbers. ASCs proliferate rapidly with a high cellular activity, making them an ideal source for obtaining MSCs.

By “adipose tissue” is meant any fat tissue. The adipose tissue may be brown or white adipose tissue, derived from subcutaneous, omental/visceral, mammary, gonadal, or other adipose tissue site. Typically, the adipose tissue is subcutaneous white adipose tissue. Such cells may comprise a primary cell culture or an immortalized cell line. The adipose tissue may be from any organism having fat tissue. Typically, the adipose tissue is mammalian, most typically the adipose tissue is human. A convenient source of adipose tissue is from liposuction surgery, however, the source of adipose tissue or the method of isolation of adipose tissue is not critical to the invention.

The preferred ASCs are the human allogeneic adipose-derived stem cells (human eASCs) authorised in the product “Darvadstrocel” (tradename “Alofisel®”). These expanded ASCs express the cell surface markers CD29, CD73, CD90 and CD105. The cells are capable of expressing factors such as vascular endothelial growth factor (VEGF), transforming growth factor-beta 1 (TGF-β1), interleukin 6 (IL-6), matrix metalloproteinase inhibitor-1 (TIMP-1) and interferon-gamma (IFN-γ) and inducible indoleamine 2,3-dioxygenase (IDO). Thus, the population of ASCs may be characterised in that at least about 50%, at least about 60%; at least about 70%; at least about 80%; at least about 85%; at least about 90% or at least about 95% or more express one or more of CD29, CD73, CD90 and/or CD105. The population of ASCs may be characterised in that at least about 50%, at least about 60%; at least about 70%; at least about 80%; at least about 85%; at least about 90% or at least about 95% of the population of cells express all of CD29, CD73, CD90 and CD105. Typically, the population of ASCs may be characterised in that at least about 80% of the population of cells express all of CD29, CD73, CD90 and CD105.

According to Bourin et al. (Cytotherapy (2013) 15(6): 641-648), a population of ASCs may be defined as being positive for expression of CD13, CD29, CD44, CD73, CD90 and CD105, and negative for expression of CD31 and CD45. In the population of ASCs, at least about 50%, at least about 60%; at least about 70%; at least about 80%; at least about 85%; at least about 90% or at least about 95% of the population of cells may express CD13, CD29, CD44, CD73, CD90 and CD105, and fewer than about 5%, about 4%, about 3% or about 2% of the population of ASCs may express CD31 and CD45. Typically, in the population of ASCs, at least about 80% of the population of cells may express CD13, CD29, CD44, CD73, CD90 and CD105, and fewer than about 5% of the population of ASCs may express CD31 and CD45.

The ASCs may be adherent to plastic under standard culture conditions. Expanded ASC (eASC) exhibit a fibroblast-like morphology in culture. Specifically, these cells are big and are morphologically characterised by a shallow cell body with few cell projections that are long and thin. The nucleus is large and round with a prominent nucleolus, giving the nucleus a clear appearance. Most of eASCs display this spindle-shaped morphology, but it is usual that some of the cells acquire polygonal morphologies (Zuk et al. Tissue Eng (2001) 7(2): 211-228).

The ASCs may be positive for the surface markers HLA I, CD29, CD44, CD59, CD73, CD90, and CD105. In some embodiments, the population of ASCs may be characterised in that at least about 50%, at least about 60%, at least about 70%, at least about 80%, at least about 85%; at least about 90% or at least about 95% of the population of ASCs express the surface markers HLA-I, CD29, CD44, CD59, CD73, CD90, and CD105. Typically, at least about 80% of the eASCs express the surface markers HLA 1, CD29, CD44, CD59, CD73, CD90, and CD105.

The ASCs may be negative for the surface markers HLAII, CD11b, CD11c, CD14, CD45, CD31, CD80 and CD86. In some embodiments, the population of ASCs may be characterised in that fewer than about 5% of the population of ASCs express the surface markers HLAII, CD11b, CD11c, CD14, CD45, CD31, CD80 and CD86. More typically, fewer than about 4%, 3% or 2% of the population of ASCs express the surface markers HLAII, CD11b, CD11c, CD14, CD45, CD31, CD80 and CD86. In one embodiment, fewer than about 1% of the population of ASCs express the surface markers HLAII, CD11b, CD11c, CD14, CD45, CD31, CD80 and CD86. In some cases, in a population of ASCs at least about 80% of the population of cells express all of CD29, CD73, CD90 and CD105 and fewer than about 5% of the population of ASCs express the surface markers HLAII, CD11b, CD11c, CD14, CD45, CD31, CD80 and CD86.

In some embodiments the population of ASCs may express one or more (e.g. two or more, three or more, four or more, five or more, six or seven) of HLA I, CD29, CD44, CD59, CD73, CD90, and CD105. In some embodiments, the eASCs may not express one or more (e.g. two or more, three or more, four or more, five or more, six or more, seven or eight) of HLAII, CD11b, CD11c, CD14, CD45, CD31, CD80. In some embodiments, the eASCs express four or more of HLA I, CD29, CD44, CD59, CD73, CD90, and CD105 and do not express four or more of HLAII, CD11b, CD11c, CD14, CD45, CD31, CD80.

Expression of CD34 may be negative or low, e.g. expressed by 0 to about 30% of the population of ASCs. Thus, in some cases, the ASCs as described above may express CD34 at low levels, e.g. in about 5 to about 30% of the population. Alternatively, in other cases, the ASCs as described do not express CD34, e.g. fewer than about 5% of the population of ASCs express CD34.

In some embodiments, the population of ASCs (e.g. at least about 50%, at least about 60%, at least about 70%, at least about 80%, at least about 85%; at least about 90% or at least about 95% of the population of cells) may express one or more (e.g. two or more, three or more, four or more, five or more, six or more, seven or more, eight or more, nine or more, or ten or more (e.g. up to 13)) of the markers CD9, CD10, CD13, CD29, CD44, CD49A, CD51, CD54, CD55, CD58, CD59, CD90 and CD105. For example, the ASCs may express one or more (e.g. two, three or all) of the markers CD29, CD59, CD90 and CD105, e.g. CD59 and/or CD90.

In some embodiments the population of ASCs may not express one or more (e.g. two or more, three or more, four or more, five or more, six or more, seven or more, eight or more, nine or more, or ten or more (e.g. up to 15)) of the markers Factor VIII, alpha-actin, desmin, S-100, keratin, CD11b, CD11c, CD14, CD45, HLAII, CD31, CD45, STRO-1 and CD133, e.g. the ASCs do not express one or more (e.g. two, three or all) of the markers CD45, CD31 and CD14, e.g. CD31 and/or CD45.

In certain embodiments, the ASCs as described above (i) do not express markers specific for antigen presenting cells (APCs); (ii) do not express IDO constitutively; and/or (iii) do not significantly express MHC II constitutively. Typically expression of IDO or MHC II may be induced by stimulation with IFN-γ. In certain embodiments, the ASCs as described above do not express Oct4.

Methods of Preparing Populations of ASCs

Methods for the isolation and culture of ASCs to provide eASCs and population of stem cells of the invention, and compositions comprising populations of stem cells populations of the invention are known in the art. ASCs are typically prepared from the stromal fraction of adipose tissue and are selected by adherence to a suitable surface e.g. plastic. Thus, the methods of stem cell cryopreservation disclosed herein may comprise an initial step (prior to step (a) of any one the methods) of: (i) isolating a population of ASCs from the stromal fraction of adipose tissue obtained from a patient, and (ii) culturing the population of ASCs. The ASCs can optionally be selected at step (i) for adherence to a suitable surface e.g. plastic. Optionally the phenotype of the ASCs may be assessed during and/or subsequent to the culturing step (ii).

Methods of Preparing Populations of ASCs

Methods for the isolation and culture of ASCs to provide eASCs and population of stem cells of the invention, and compositions comprising populations of stem cells populations of the invention are known in the art. ASCs are typically prepared from the stromal fraction of adipose tissue and are selected by adherence to a suitable surface e.g. plastic. Thus, the methods of stem cell cryopreservation disclosed herein may comprise an initial step (prior to step (a) of any one the methods) of: (i) isolating a population of ASCs from the stromal fraction of adipose tissue obtained from a patient, and (ii) culturing the population of ASCs. The ASCs can optionally be selected in step (i) for adherence to a suitable surface e.g. plastic. Optionally, the phenotype of the ASCs may be assessed during and/or subsequent to the culturing step (ii).

ASCs can be obtained by any means standard in the art. Typically said cells are obtained disassociating the cells from the source tissue (e.g. lipoaspirate or adipose tissue), typically by treating the tissue with a digestive enzyme such as collagenase. The digested tissue matter is then typically filtered through a filter of between about 20 microns to 1 mm. The cells are then isolated (typically by centrifugation) and cultured on an adherent surface (typically tissue culture plates or flasks). Such methods are known in the art and e.g. as disclosed in U.S. Pat. No. 6,777,231.

According to this methodology, lipoaspirates are obtained from adipose tissue and the cells derived therefrom. In the course of this methodology, the cells may be washed to remove contaminating debris and red blood cells, preferably with PBS. The cells are digested with collagenase (e.g. at 37° C. for 30 minutes, 0.075% collagenase; Type I, Invitrogen, Carlsbad, Calif.) in PBS. To eliminate remaining red blood cells, the digested sample can be washed (e.g. with 10% fetal bovine serum),

treated with 160 mmol/L NH₄Cl, and finally suspended in DMEM complete medium (DMEM containing 10% FBS, 2 mM glutamine and 1% penicillin/streptomycin). The cells can be filtered through a 40 μm nylon mesh.

Cultured human ASCs are described in DelaRosa et al. (Tissue Eng Part A. (2009) 15(10): 2795-806), Lopez-Santalla et al. (Stem cells (2015) 33: 3493-3503). In one embodiment (as described in Lopez-Santalla et al. (2015), cited above), human adipose tissue aspirates from healthy donors were washed twice with phosphate-buffered saline and digested with 0.075% collagenase (Type I; Invitrogen). The digested sample was washed with 10% fetal bovine serum (FBS), treated with 160 mM NH₄Cl to eliminate the remaining erythrocytes, and suspended in culture medium (Dulbecco's modified Eagle's medium (DMEM) with 10% FBS). Cells were seeded (2-3×104 cells/cm²) in tissue culture flasks and cultured (37° C., 5% CO₂) with change of culture medium every 3-4 days. Cells were transferred to a new flask (10³ cells/cm²) when they reached 90% confluence. Cells were expanded up to duplication 12-14 and frozen.

In another embodiment (as described by DelaRosa et al. (2009), Tissue Eng Part A 15(10): 2795-806), lipoaspirates obtained from human adipose tissue from healthy adult donors were washed twice with PBS, and digested at 37° C. for 30 minutes with 18 U/mL of collagenase type I in PBS. One unit of collagenase liberates 1 mM of L-leucine equivalents from collagen in 5 hours at 37° C., pH 7.5 (Invitrogen, Carlsbad, Calif.). The digested sample was washed with 10% of fetal bovine serum (FBS), treated with 160 mM NH₄Cl, suspended in culture medium (DMEM containing 10% FBS), and filtered through a 40-mm nylon mesh. Cells were seeded (2-3×10⁴ cells/cm²) onto tissue culture flasks and expanded at 37° C. and 5% CO₂, changing the culture medium every 7 days. Cells were passed to a new culture flask when cultures reached 90% of confluence. Cells were phenotypically characterized by their capacity to differentiate into chondro-, osteo-, and adipogenic lineages.

The ASCs are cultured in a suitable tissue culture vessel comprising a surface suitable for the adherence of ASCs, e.g. plastic. Non-adherent cells are removed, e.g. by washing in a suitable buffer, to provide an isolated population of adherent stromal cells (e.g. ASC). Cells isolated in this way can be seeded (preferably 2-3×10⁴ cells/cm²) onto tissue culture flasks and expanded at 37° C. and 5% CO₂, changing the culture medium every 3-4 days. Cells are preferably detached from the adherent surface (e.g. by means of trypsin) and passed (“passaged”) to a new culture flask (1,000 cells/cm²) when cultures reach around 90% of confluence.

The ASCs may be cultured for at least about 15, at least about 20 days, at least about 25 days, or at least about 30 days. Typically the expansion of cells in culture improves the homogeneity of the cell phenotype in the population, such that a substantially pure population is obtained.

In some embodiments, the ASCs are expanded in culture for at least three culture passages or “passaged at least three times.” In other embodiments, the cells are passaged at least four times, at least five times, at least six times, at least seven times, at least eight times, at least nine times, or at least ten times. It is preferable that cells are passaged more than three times to improve the homogeneity of the cell phenotype in the cell population. Indeed, the cells may be expanded in culture indefinitely so long as the homogeneity of the cell phenotype is improved and differential capacity is maintained.

In some embodiments, the ASC are multiplied in culture for at least three population doublings, for example, the cells are expanded in culture for at least four, five, six, seven, eight, nine, ten, 15 or 20 population doublings. In some embodiments, the cells are expanded in culture for less than seven, eight, nine, ten, 15 or 20 population doublings. In certain embodiments, the cells are expanded in culture for between about 5 and 10 population doublings. In certain embodiments, the cells are expanded in culture for between about 10 and 15 population doublings. In certain embodiments, the cells are expanded in culture for between about 15 and 20 population doublings, for example about 16 population doublings. ASC isolation is preferably carried out under sterile or GMP conditions.

“Allogeneic therapy” herein means a cell-based therapy, wherein the donor is a different person to the recipient of the cells. Preferably, the allogeneic therapy does not involve HLA matching between donors and recipients. In pharmaceutical manufacturing, the allogeneic methodology is promising, because HLA-unmatched allogeneic therapies can form the basis of “off the shelf” products.

“Presensitization” herein means that the patients show a pre-existing immunity for a given SC population before the actual administration of the SC population. The pre-existing immunity may be associated with the presence of donor-specific antibodies (DSA), in particular DSA being specific for HLA-Class I molecules. This can be determined by methods known in the art such as spectralphotometric, flow cytometry crossmatch (FCXM), fluorescent or luminescent assays (e.g.ELISA (Enzyme-linked immunosorbent assay), wherein the HLA-Class I molecule is bound to the plate. The pre-existing immunity may e.g. be due to earlier blood transfusions or in women due to earlier pregnancies.

“Retreatment” herein means that after administration of a first dose of a first SC population from a first donor to a patient the patient either receives another dose of the first SC population from the first donor or a dose of a second SC population from a second donor. Retreatment may be necessary to increase the treatment efficacy compared to a single dose administration.

“Culturing a sample of an SC population” means herein that the SC population is contained in a cell culture medium suitable for maintaining viability of the SC population. Suitable cell culture media may contain essential nutrients for the SC population such as amino acids, carbohydrates, vitamins and/or salts. The pH is adapted to suitable conditions by the use of buffers. Preferably, the cell culture medium is selected from RPMI (available from Gibco) or DMEM (available from e.g. Sigma-Aldrich). “An IFN-γ concentration capable of inducing maximal HLA-Class I expression in said SC population” means herein an IFN-γ concentration which is capable of providing the maximal amount of HLA-Class I molecules on the surface of said SC population. The amount of HLA-Class I molecules on the surface of said SC population can be determined by Fluorescence-activated cell sorting (FACS) or in an ELISA assay format such as in a sandwich assay format using an HLA-Class I antibody either directly linked to a chromophore or fluorophore or indirectly with a labelled secondary antibody. Maximal HLA-Class I expression is induced, if the expression of HLA-Class I does not increase further when the cells are treated with a higher IFN-γ concentration.

IFN-γ is known to increase the expression and presentation of HLA-Class I molecules. According to a preferred embodiment the IFN-γ concentration capable of inducing maximal HLA-class I expression in said SC population is from about 0.5 to about 30 ng/ml, preferably from about 1 to about 15 ng/ml, more preferably from about 2 to about 4 ng/ml and most preferably it is 3 ng/ml. Preferably, the IFN-γ is incubated with the SC population for a time period of 12 to 72 hours, preferably for a time period of 24 to 60 hours, more preferably for a time period of 30 to 54 hours and most preferably for a time period of 48 hours. Most preferably, the IFN-γ concentration capable of inducing maximal HLA-class I expression in said SC population is 3 ng IFN-γ/ml and the IFN-γ is incubated with the SC populationfor a time period of 48 hours.

“An HLA-Class I antibody” means herein an antibody capable of specifically binding to HLA-A, HLA-B and/or HLA-C. “Specifically” in this context shall mean that the binding is not unspecific. The term “specifically” includes binding to antigens sharing epitopes which allow cross-reactivity of the antibodies also to related antigens. The antibody may be of any subtype including IgG and IgM. The antibody may be from any source, preferably the antibody is a murine, rabbit, sheep or goat antibody, preferably a murine antibody. The antibody preferably includes a constant region (Fc region) capable of interacting with proteins of the complement system.

In a preferred embodiment the HLA-Class I antibody specifically binds to HLA-A, HLA-B and HLA-C. It is therefore preferred that the antibody is a pan-HLA-Class I antibody recognizing an epitope being conserved among HLA-A, HLA-B and HLA-C heavy chains.

In a more preferred embodiment the HLA-Class I antibody has essentially the same binding affinity for HLA-A, preferably HLA-A2, most preferred HLA-A*0201, as the antibody produced by the hybridoma clone w6/32 obtainable from ATCC (designation: HB-95) or ECACC (No.: 84112003). Preferably, the binding affinity is determined by using an ELISA method. “Essentially the same binding affinity” means that the binding affinity of the HLA-Class I antibody differs by less than 10%, preferably less than 8% and more preferably less than 5% from the binding affinity of the antibody produced by the hybridoma clone w6/32.

In an even more preferred embodiment the antibody is produced by the hybridoma clone w6/32. This hybridoma clone is obtainable from ATCC (designation: HB-95) or ECACC (No.: 84112003).

“A range of different concentrations of an HLA-Class I antibody” means herein that the test sample and the control sample is treated with different concentrations of the HLA-Class I antibody resulting in different CDC values as determined by cell lysis. The different CDC values can be depicted in a test curve dependent on the concentration of the HLA-Class I antibody.

In one embodiment the different concentrations of the HLA-Class I antibody are within the range of from about 1 to about 50 ng/ml. In one embodiment, two or three different concentrations of the HLA-Class I antibody within the range of from about 1 to about 50 ng/ml are used. In one embodiment, four or five different concentrations of the HLA-Class I antibody within the range of from about 1 to about 50 ng/ml are used. In one embodiment, six or seven different concentrations of the HLA-Class I antibody within the range of from about 1 to about 50 ng/ml are used. In one embodiment, eight or nine different concentrations of the HLA-Class I antibody within the range of from about 1 to about 50 ng/ml are used.

In one embodiment the different concentrations of the HLA-Class I antibody produced by the hybridoma clone w6/32 are within the range of from about 1 to about 50 ng/ml. In one embodiment, two or three different concentrations of the HLA-Class I antibody produced by the hybridoma clone w6/32 within the range of from about 1 to about 50 ng/ml are used. In one embodiment, four or five different concentrations of the HLA-Class I antibody produced by the hybridoma clone w6/32 within the range of from about 1 to about 50 ng/ml are used. In one embodiment, six or seven different concentrations of the HLA-Class I antibody produced by the hybridoma clone w6/32 within the range of from about 1 to about 50 ng/ml are used. In one embodiment, eight or nine different concentrations of the HLA-Class I antibody produced by the hybridoma clone w6/32 within the range of from about 1 to about 50 ng/ml are used.

In a further preferred embodiment the range of different concentrations of the HLA-Class I antibody is at least two or three, preferably four or five, more preferably six or seven and most preferably seven or eight concentrations selected from 1 ng/ml, 3 ng/ml, 5 ng/ml, 10 ng/ml, 15 ng/ml, 20 ng/ml, 30 ng/ml, 40 ng/ml and 50 ng/ml.

“Under conditions such that the HLA-Class I antibody binds to HLA-Class I expressed in the test sample and the control sample” means herein that the SC population of the test sample and the control sample is contained in a suitable medium which allows the binding of the antibody to the stem cells. The medium therefore has a pH and salt concentration which does not denature the antibody and/or the cells. Preferably, the medium contains a suitable buffer systems for providing a pH similar to physiological pH in human blood (pH 7.35 to 7.45). More preferably the medium is a phosphate-buffered saline (PBS) solution having a pH of 7.4 and containing 137 mM NaCl, 2.7 mM KCl, 10 mM Na₂HPO₄, 1.8 mM KH₂PO₄.

“Complement” as used herein means complement derived from any source of blood including plasma or serum. The complement may also be used as mixtures of purified or synthesized complement proteins. The complement system consists of a number of small proteins found in the blood, which are synthesized by the liver and circulate as inactive precursors. When stimulated by one of several triggers, proteases in the system cleave specific proteins to release cytokines and initiate an amplifying cascade of further cleavages. The end result of this complement activation or complement fixation cascade is stimulation of phagocytes to clear foreign and damaged material, inflammation to attract additional phagocytes, and activation of the cell-killing membrane attack complex (MAC) which results in cell lysis of the attacked cells. During allogeneic recognition, complement-dependent cytotoxicity (CDC) is initiated when C1q, the initiating mediator of the classical pathway, is bound to the Fc portion of HLA Class-I antigen-bound antibodies resulting in the activation of C1q and subsequent complement cascade signalling.

“Saturated with complement” herein means that the amount of complement added in the method according to the invention is in molar excess to the amount of HLA-Class I antibody bound on the surface of the IFN-γ induced SC population (tested sample). Saturation with complement can experimentally be determined by performing the assay using different concentrations of complement. Saturation is achieved once the CDC activity has reached its maximum and can no longer be increased by the addition of higher concentrations of complement.

In a preferred embodiment the complement used in step (c) of claim 1 is from serum. Preferably, the serum has not been treated with heat. Heat-inactivation may denature the complement proteins rendering them incapable of forming the MAC complex. Also preferably, the serum is not fetal bovine serum. More preferably, the serum is from rabbit, goat or sheep. Most preferably, the serum is from rabbit. Such a serum is obtainable from One Lambda.

In a preferred embodiment the serum concentration resulting in saturation of the bound HLA-I antibody with complement is from 50 to 83.33% (v/v), preferably the serum concentration serum concentration resulting in saturation of the bound HLA-I antibody with complement is from 66.66 to 83.33% (v/v), more preferably the serum concentration resulting in saturation of the bound HLA-I antibody with complement is from 75 to 83,33% (v/v) and most preferably the serum concentration serum concentration resulting in saturation of the bound HLA-I antibody with complement is 83.33% (v/v).

“Complement-dependent cytotoxicity (CDC)” is an effector function of IgG and IgM antibodies. When they are bound to surface antigen, the classical complement pathway is triggered, resulting in formation of the membrane attack complex (MAC) and target cell lysis. In the context of the present invention CDC is determined by measuring the cell lysis induced in the SC population by binding of HLA-Class I antibodies to HLA Class I molecules expressed on the SC population.

In a preferred embodiment the cell lysis is determined by measuring a chemiluminescent or fluorescent dye selective for viable or lysed cells or a radioactive agent released from lysed cells. Viable cells with intact cell membranes may be distinguished from lysed cells by methods known in the art using different types of agents.

As a radioactive method for distinguishing viable cells from dead cells, the Chromium release assay has been established. As an alternative to radioactive assays, fluorescent dyes have been developed. For example, non-fixable viability dyes for DNA binding such as propidium iodide, DAPI, 7-aminoactinomycin D (7-AAD), TO-PRO-3 may be used. When fixation of sample is required amine reactive dyes from Invitrogen or Fixable Viability Dyes from eBioscience (eFluor450, eFluor660, eFluor780) or Violet fixable dye from Becton Dickinson (BD Horizon VD450) can be added to samples to discriminate between live and dead cells prior to fixation. Further dyes are available suitable for assessment of cell viability by cell functionality such as dyes requiring esterase activity (e.g. Calcein AM from eBioscience/Invitrogen) or dyes accumulating in mitochondria (JC-1, Rhodamine 123).

As a further alternative, chemiluminescence-based assays or bioluminescence-based assays have been developed (e.g. the menadione-catalyzed H₂O₂ production). All of them are contemplated by the present invention for determining the CDC by measuring the cell lysis. In a preferred embodiment the CDC acitivity is determined by using 7-aminoactinomycin D as dye in a FACS analysis as further described in the examples.

The “EC₅₀ value” as defined in the claims is the concentration of HLA-Class I antibody that induces 50% of the maximal CDC (EC₅₀ value) in the test sample and the control sample. “Determining the EC₅₀ value” may be done visually or with a commercially available software for biological data analysis using the determined CDC values for the different HLA-Class I antibody concentrations. Software capable of linear regression analysis may be used. A suitable software may be FCS Express Version 5 (DeNovo Software).

In a preferred embodiment the SC population for allogeneic therapy is selected according to step f1) if the ratio of the EC₅₀ value of the control sample to the EC₅₀ value of the test sample is between about 0.1 to about 1.25, preferably between about 0.1 and about 1.0, more preferably between about 0.1 and about 0.5, particularly preferably between about 0.1 and about 0.25.

In a further preferred embodiment the SC population for allogeneic therapy is selected according to step f2) if the EC₅₀ value of the test sample is between about 3.5 ng/ml to about 30 ng/ml, preferably between about 9 ng/ml to about 25 ng/ml, more preferably between about 10 ng/ml to about 20 ng/ml of the HLA-Class I antibody.

The “CD46 expression level” may be determined on the nucleic acid level by standard techniques in the art such as Northern Blot and RT-qPCR. The CD46 mRNA sequence is available from public databases (NCBI Reference sequence: NM_002389.4). Suitable primers may be prepared by methods known in the art. The CD46 expression level may also be determined on the protein level. Suitable methods include Western Blot and FACS analysis using labelled HLA-Class I antibody molecules.

According to a further aspect is provided an SC population suitable for allogeneic therapy, in particular for the treatment of presensitized patients or retreatment with allogeneic therapy, having any of the following properties:
i) a ratio of the EC₅₀ value of the control sample to the EC₅₀ value of the test sample of less than 1.25, preferably less than 1.0, more preferred less than 0.5 and particularly preferred less than 0.25, wherein the EC₅₀ value is determined according to the invention;
ii) an EC₅₀ value of the test sample of at least 3.5 ng/ml HLA-Class I antibody, preferably at least 9 ng/ml, more preferred at least 15 ng/ml, particularly preferred at least 20 ng/ml HLA-Class I antibody, wherein the EC₅₀ value is determined according to the invention; or
iii) a ratio of CD46 expression in the test sample to the CD46 expression in the control sample is more than 2.0, preferably more than 2.5, particularly preferred more than 3.0, wherein the CD46 expression is determined according to the invention.

In a preferred embodiment the SC population is selected by the method according to the invention. Also preferred the SC population is a mesenchymal stem cell (MSC) population, preferably a BM-MSC population or an ASC population, preferably a human BM-MSC or a human ASC.

According to a further aspect is provided a pharmaceutical composition comprising the SC population according to the invention and optionally a pharmaceutically acceptable carrier.

The term “pharmaceutically acceptable carrier” herein means any pharmaceutically acceptable carrier known in the art which does not adversely affect the viability or efficacy of the SC cell population. The pharmaceutically acceptable carrier may be a cell culture medium containing amino acids, salts and vitamins. Preferably, the cell culture medium is RPMI or DMEM, more preferably the cell culture medium is DMEM. The cell culture medium may be supplemented with human serum albumin (HSA) in a concentration from about 5% (v/v) to about 30% (v/v), more preferably, the HSA concentration may be 20% (v/v). In one embodiment the pharmaceutically acceptable carrier is DMEM with 20% (v/v) HSA.

The pharmaceutical composition may be in the form of a suspension for injection. The concentration of the SC population in the suspension may be in the range from 1×10⁵ to 8×10⁶ cells/ml, preferably from 1×10⁶ to 6×10⁶ cells/ml. Most preferably, the concentration is 5×10⁶ cells/ml. The pharmaceutical composition may be administered by injection.

According to a further aspect is provided by a method for preparing a pharmaceutical composition comprising the steps:
a) performing the method of according to the invention;
b) formulating the selected SC population with at least one pharmaceutically acceptable carrier.

In a further aspect the SC population according to the invention is provided for use in allogeneic stem cell therapy in a patient in need thereof, preferably in a presensitized patient or a patient undergoing retreatment. In a more preferred embodiment, the patient in need suffers from a disease selected from fistulas, leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease, preferably the disease is a fistula, more preferably the disease is a complex perianal fistula.

According to a further aspect is provided an allogeneic stem cell therapy method comprising administering the SC population according to the invention to a patient in need thereof, preferably in a presensitized patient or a patient undergoing retreatment. The patient may suffer from any of the diseases mentioned above.

In one embodiment the present invention relates to an in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample), wherein the SC population is an ASC population and the IFN-γ concentration is 3 ng/ml applied over 48 hours;
b) contacting the test sample and the control sample with a range of different concentrations of an HLA-Class I antibody under conditions such that the HLA-Class I antibody binds to HLA-Class I expressed in the test sample and the control sample, wherein the HLA-Class I antibody is w6/32 and wherein the antibody concentrations are 1 ng/ml, 3 ng/ml, 5 ng/ml, 10 ng/ml, 15 ng/ml, 20 ng/ml, 30 ng/ml, 40 ng/ml and 50 ng/ml;
c) adding complement to the test sample and the control sample such that the bound HLA-Class I antibody is saturated with complement and complement-dependent cytotoxicity (CDC) is induced, wherein the complement is added in the form of rabbit serum;
d) determining the CDC for the range of different concentrations of the HLA-Class I antibody by measuring the cell lysis induced in the test sample and the control sample;
e) determining the concentration of HLA-Class I antibody that induces 50% of the maximal CDC (EC₅₀ value) in the test sample and the control sample; and
f1) selecting the SC population for allogeneic therapy if the ratio of the EC₅₀ value of the control sample to the EC₅₀ value of the test sample is less than 1.25, preferably less than 1.0, more preferably less than 0.5; particularly preferably less than 0.25; or
f2) selecting the SC population for allogeneic therapy if the EC₅₀ value of the test sample is at least 3.5 ng/ml, preferably at least 9 ng/ml, more preferably at least 15 ng/ml, particularly preferably at least 20 ng/ml of the HLA-Class I antibody.

In one embodiment the present invention relates to an in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample), wherein the SC population is an ASC population and the IFN-γ concentration is 3 ng/ml applied over 48 hours;
b) contacting the test sample and the control sample with a range of different concentrations of an HLA-Class I antibody under conditions such that the HLA-Class I antibody binds to HLA-Class I expressed in the test sample and the control sample, wherein the HLA-Class I antibody is w6/32 and wherein the antibody concentrations are 1 ng/ml, 3 ng/ml, 5 ng/ml, 10 ng/ml, 15 ng/ml, 20 ng/ml, 30 ng/ml, 40 ng/ml and 50 ng/ml;
c) adding complement to the test sample and the control sample such that the bound HLA-Class I antibody is saturated with complement and complement-dependent cytotoxicity (CDC) is induced, wherein the complement is added in the form of rabbit serum;
d) determining the CDC for the range of different concentrations of the HLA-Class I antibody by measuring the cell lysis induced in the test sample and the control sample;
e) determining the concentration of HLA-Class I antibody that induces 50% of the maximal CDC (EC₅₀ value) in the test sample and the control sample; and
f1) selecting the SC population for allogeneic therapy if the ratio of the EC₅₀ value of the control sample to the EC₅₀ value of the test sample between about 0.1 to about 1.25, preferably between about 0.1 and about 1.0, more preferably between about 0.1 and about 0.5, particularly preferably between about 0.1 and about 0.25; or
f2) selecting the SC population for allogeneic therapy if the EC₅₀ value of the test sample is between about 3.5 ng/ml to about 30 ng/ml, preferably between about 9 ng/ml to about 25 ng/ml, more preferably between about 10 ng/ml to about 20 ng/ml of the HLA-Class I antibody.

Some Embodiments of the Present Invention Relate to:
1. An in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample);
b) contacting the test sample and the control sample with a range of different concentrations of an HLA-Class I antibody under conditions such that the HLA-Class I antibody binds to HLA-Class I expressed in the test sample and the control sample;
c) adding complement to the test sample and the control sample such that the bound HLA-Class I antibody is saturated with complement and complement-dependent cytotoxicity (CDC) is induced;
d) determining the CDC for the range of different concentrations of the HLA-Class I antibody by measuring the cell lysis induced in the test sample and the control sample;
e) determining the concentration of HLA-Class I antibody that induces 50% of the maximal CDC (EC₅₀ value) in the test sample and the control sample; and
f1) selecting the SC population for allogeneic therapy if the ratio of the EC₅₀ value of the control sample to the EC₅₀ value of the test sample is less than 1.25, preferably less than 1.0, more preferably less than 0.5; particularly preferably less than 0.25; or
f2) selecting the SC population for allogeneic therapy if the EC₅₀ value of the test sample is at least 3.5 ng/ml, preferably at least 9 ng/ml, more preferably at least 15 ng/ml, particularly preferably at least 20 ng/ml of the HLA-Class I antibody.
2. The in vitro method of item 1, wherein the method further comprises the steps of determining the CD46 expression level in the test sample and in the control sample; and selecting the SC population for allogeneic therapy if the ratio of CD46 expression in the test sample to the CD46 expression in the control sample is more than 2.0, preferably more than 2.5, particularly preferably more than 3.0.
3. The in vitro method of item 1 or 2, wherein the SC population is a mesenchymal stem cell (MSC) population, preferably a human MSC population.
4. The in vitro method of item 3, wherein the mesenchymal stem cell population is a bone-marrow-derived stem cell (BM-MSC) population.
5. The in vitro method of item 3, wherein the mesenchymal stem cell population is an adipose tissue-derived stem cell (ASC) population.
6. The in vitro method of item 5, wherein the ASC population expresses CD29, CD73, CD90 and/or CD105.
7. The in vitro method of any one of items 1 to 6, wherein the IFN-γ concentration capable of inducing maximal HLA-class I expression in said SC population is from about 0.5 to about 30 ng/ml, preferably from about 1 to about 15 ng/ml, more preferred from about 2 to about 4 ng/ml.
8. The in vitro method of item 7, wherein the IFN-γ concentration capable of inducing maximal HLA-class I expression in said SC population is 3 ng IFN-γ/ml, preferably applied over a time period of 48 hours.
9. The in vitro method of any one of items 1 to 8, wherein the HLA class I antibody specifically binds to HLA-A, HLA-B and/or HLA-C.
10. The in vitro method of item 9, wherein the HLA class I antibody specifically binds to HLA-A, HLA-B and HLA-C, preferably the HLA class I antibody is a murine monoclonal antibody.
11. The in vitro method of item 9 or 10, wherein the HLA-Class I antibody has essentially the same binding affinity for HLA-A as the antibody produced by the hybridoma clone w6/32 obtainable from ATCC (designation: HB-95) or ECACC (No.: 84112003).
12. The in vitro method of any one of items 9 to 11, wherein the antibody is produced by the hybridoma clone w6/32 obtainable from ATCC (designation: HB-95) or ECACC (No.: 84112003).
13. The in vitro method of any one of items 1 to 12, wherein two or three different concentrations of the HLA-Class I antibody within the range of from about 1 to about 50 ng/ml are used, preferably four or five different concentrations of the HLA-Class I antibody within the range of from about 1 to about 50 ng/ml are used, more preferably six or seven different concentrations of the HLA-Class I antibody within the range of from about 1 to about 50 ng/ml are used and even more preferably eight or nine different concentrations of the HLA-Class I antibody within the range of from about 1 to about 50 ng/ml are used.
14. The in vitro method of item 13, wherein the range of different concentrations of the HLA-Class I antibody is 1 ng/ml, 3 ng/ml, 5 ng/ml, 10 ng/ml, 15 ng/ml, 20 ng/ml, 30 ng/ml, 40 ng/ml and 50 ng/ml.
15. The in vitro method of any one of items 1 to 14, wherein the complement used in step (c) of claim 1 is from serum.
16. The in vitro method of claim 15, wherein the serum has not been treated with heat.
17. The in vitro method of claim 15 or 16, wherein the serum is from rabbit, goat or sheep, preferably is from rabbit.
18. The in vitro method of any one of claims 15 to 17, wherein the serum concentration is from 50 to 83.33% (v/v), preferably the serum concentration is from 66.66 to 83.33% (v/v).
19. The in vitro method of any one of items 1 to 18, wherein the cell lysis is determined by measuring a chemiluminescent or fluorescent dye selective for viable or lysed cells or a radioactive agent released from lysed cells.
20. An SC population suitable for allogeneic therapy, in particular for the treatment of presensitized patients or retreatment with allogeneic therapy, having any of the following properties:
i) a ratio of the EC₅₀ value of the control sample to the EC₅₀ value of the test sample of less than 1.25, preferably less than 1.0, more preferably less than 0.5 and particularly preferably less than 0.25, wherein the EC₅₀ value is determined as set forth in item 1;
ii) an EC₅₀ value of the test sample of at least 3.5 ng/ml HLA-Class I antibody, preferably at least 9 ng/ml, more preferably at least 15 ng/ml, particularly preferably at least 20 ng/ml HLA-Class I antibody, wherein the EC₅₀ value is determined as set forth in item 1; and/or
iii) a ratio of CD46 expression in the test sample to the CD46 expression in the control sample is more than 2.0, preferably more than 2.5, particularly preferably more than 3.0, wherein the CD46 expression is determined as set forth in item 2.
21. The SC population according to item 20, wherein the SC population is selected by the method of any one of items 1 to 19.
22. The SC population of item 21 or 22, wherein the SC population is a mesenchymal stem cell (MSC) population, preferably a BM-MSC population or an ASC population, preferably a human BM-MSC or a human ASC.
23. A pharmaceutical composition comprising the SC population according to any one of items 20 to 22 and optionally a pharmaceutically acceptable carrier.
24. A method for preparing a pharmaceutical composition comprising the steps:
a) performing the method of any one of items 1 to 19;
b) formulating the selected SC population with at least one pharmaceutically acceptable carrier.
25. The SC population of any one of items 20 to 22 for use in allogeneic stem cell therapy in a patient in need thereof, preferably in a presensitized patient or a patient undergoing retreatment.
26. The SC population for use according to item 25, wherein the patient in need suffers from a disease selected from fistulas, leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease, preferably the disease is a fistula, more preferably the disease is a complex perianal fistula.
27. An allogeneic stem cell therapy method comprising administering the SC population of any one of items 20 to 22 to a patient in need thereof, preferably in a presensitized patient or a patient undergoing retreatment.
28. The allogeneic stem cell therapy method of item 27, wherein the patient in need suffers from a disease selected from leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease, preferably the disease is a fistula, more preferably the disease is a complex perianal fistula.

The invention is further described in the following examples which are solely for the purpose of illustrating specific embodiments of the invention, and are also not to be construed as limiting the scope of the invention in any way.

### EXAMPLES

### Material and Methods

### Monitoring DSA Generation in ADMI CD1

Patients

A subgroup of 123 patients from the randomized, double-blind, parallel-group, placebo-controlled study, ADMIRE CD1 (Panes et al. (2016), Lancet 388, 1281-1290). Briefly, all were adult patients (≥18 years) with CD and treatment-refractory draining, complex perianal fistulas and were selected to receive a single intra-lesional injection of 120 million ASCs or 24 mL saline solution (placebo). A total of 60 and 63 patients received placebo or infusion of ASCs, respectively, from which 105 (58 ASCs, 47 placebo) were successfully followed for up to 52 weeks after administration.

ASC Donors

Human adipose tissue aspirates from healthy donors were processed as described elsewhere (Lopez-Santalla et al., 2015, cited above). For the present study ASCs from seven different donors were used; DonA was the donor used for the ADMIRE CD1 clinical trial. DonA and DonB were from the ADMIRE CD2 clinical trial (NCT03279081) (which utilized two different donors). Additionally, ASCs from donors DonC, DonD, DonE, DonF and DonG were analyzed. All ASC donors complied with the identity and purity criteria set by the International Federation for Adipose Therapeutics and the International Society for Cellular Therapy (Bourin et al., (2013), Cytotherapy (2013) 15(6): 641-648). ASC culture has been described elsewhere (DelaRosa et al., 2009, cited above).

Anti-HLA Detection

A plasma sample was obtained by centrifugation of a peripheral blood tube with ethylenediaminetetraacetic acid (Vacutainer® spry-coated K2EDTA tubes, BD), collected from all patients at baseline and at 12 and 52 weeks after placebo or ASC administration. Anti-HLA antibodies were detected in a Luminex platform using a LabscreenMixed™ kit (One Lambda Inc.® Canoga Park, Calif., US) according to manufacturer's instructions. All samples with a signal >800 units of median of fluorescence intensity (MFI) were considered positive, and specificities of HLA antibodies were determined using the Labscreen Single Antigen™ kit (One Lambda Inca Canoga Park, Calif., US). All signals were normalized according to Quantiplex™ beads fluorescence where >20,000 standard fluorescent intensity units were considered relevant. Qualitatively, the HLA antibody titer was defined as the resulting MFI sum of all determinant beads from the HLA class I molecules included in the Labscreen Mixed kit. This allowed to compare the generated level of humoral response, independently of the presented anti-HLA specificities.

HLA Typing

The assignment of HLA allele expressed in each patient was determined from DNA samples obtained from peripheral blood sample using the chemagic DNA Blood250 KIT (PerkinElmer). After checking purity via examination of the A260/280 absorbance ratio, all samples with a DNA concentration of 20 ng/μL or more were tested using a LABType® SSO assay (One Lambda, Canoga Park, Calif.) specifically for loci A, B and C of HLA, according to manufacturer instructions. The characterization of the incompatibilities between patient and donor ASCs were defined as an unshared, unique chain of polymorphic residues, using the algorithm HLA matchmaker hereafter referred to as eplets (Duquesnoy (2002), Hum Immunol 63, 339-352).

### Standardization of Flow Cytometry Crossmatch (FCXM) Binding with Recombinant Anti-HLA Antibodies (rHLA)

Standard Curves

The level of class I and class II HLA expression was determined in ASCs from DonA and DonB (class I), and DonA (class II) under basal conditions and the ASCs were pre-activated using interferon IFNγ (3 ng/mL for 48 hours). 50,000 ASCs were stained with the PE (phycoerythrin) labelled anti-human class I HLA Ab (clone W6/32) and PerCP (Peridinin-chlorophyll-protein) labelled anti-human class II HLA Ab (clone L243) (Becton Dickinson, Franklin Lakes, N.J., US) in increasing concentrations (from 0 to 15 ng/mL for clone W6/32, and 0 to 3 ng/mL for clone L243) and incubated for 30 minutes in the dark at room temperature.

Plasma Samples FCXM Binding Strength

Pre-treatment week 12 (W12) and week 52 (W52) plasma samples from all patients administered with ASCs that were previously de-complemented at 56° C. for 30 minutes and washed once with Magnetic-activated cell sorting (MACS) buffer were tested. 50 μL of de-complemented plasma were incubated with 50,000 ASCs (final volume was 100 μL) for 30 minutes at room temperature. The FACS-Fortessa X20 cell analyzer was used to determine the MFI of HLA-Class 1 and HLA-Class II by acquiring 10,000 events in P1 gate (total population of ASCs) per sample. For analysis the BD FacsDiva™ software (BD) was used.

Plasma Sample CDC Assay

For cytotoxicity measurements, 250 μL of rabbit serum as a source of complement anti-human class I HLA (CABC-1D, One Lambda Inc® Canoga Park, Calif., US) was added to the cells for 1 hour. Cells were then washed twice and incubated with 20 μL FITC anti-human IgG within 20 minutes. Finally, after washing, cytotoxicity was determined after adding 5 μL of 7-Aminoactinomycin D (7-AAD) viability dye by acquisition in a LSR Fortessa flow cytometer (BD).

mCRP Quantification Via FACS

ASCs were grown in either normal or 3 ng/mL IFNγ conditions for 48 hours. ASCs were then trypsinized and counted. A total of 50,000 cells were resuspended in 100 μL MACS buffer. For staining we used CD46 (cat number 564253, BD), CD55 (MCA1614PE, Serotec) and CD59 (BRA-10G, Novus Biologicals) Abs and their respective isotypes as controls (IgG2a-APC, IgG1-PE and IgG2b-PE from BD). After a 20-minute ice incubation, ASCs were washed with MACS and centrifuged at 1,500 rpm for 4 minutes. Finally, ASCs were re-suspended in 100 μL MACS, transferred to cytometry tubes and acquired in a LSR Fortessa flow cytometer (BD) and analyzed using BD FacsDiva™ (BD).

Generation of CD46 Knockout Canines

Guide RNA was designed to target CD46 exon 3 using the following public genomic tools: www.genome.ucsc.edu and www.www.ncbi.nlm.nih.gov/gene. For CRISPR RNA (crRNA) delivery the Alt-R® CRISPR-Cas9 System (IDT Integrated DNA Technologies) was used according to manufacturer's instructions. Briefly, ASCs were thawed and left overnight. Following this, ribonucleoprotein complex mixes were prepared and delivered using Lipofectamine™ RNAiMAX (Thermo-Fisher). crRNA and trans-activating crRNA (tracrRNA) were mixed in an equimolar concentration within a sterile micro-centrifuge tube at a final oligo duplex working concentration of 1 μM. The mixture was incubated for 20 minutes at room temperature. Following this, the transfection complexes were added to the culture plate before adding the ASC suspension. After 24 hours the ASC medium was replaced and lipofection efficacy was checked under the microscope using fluorescence tracrRNA-ATT⁵⁵⁰.

RESULTS

### Long-Term DSA Presence in ADMIRE CD1-Treated Patients

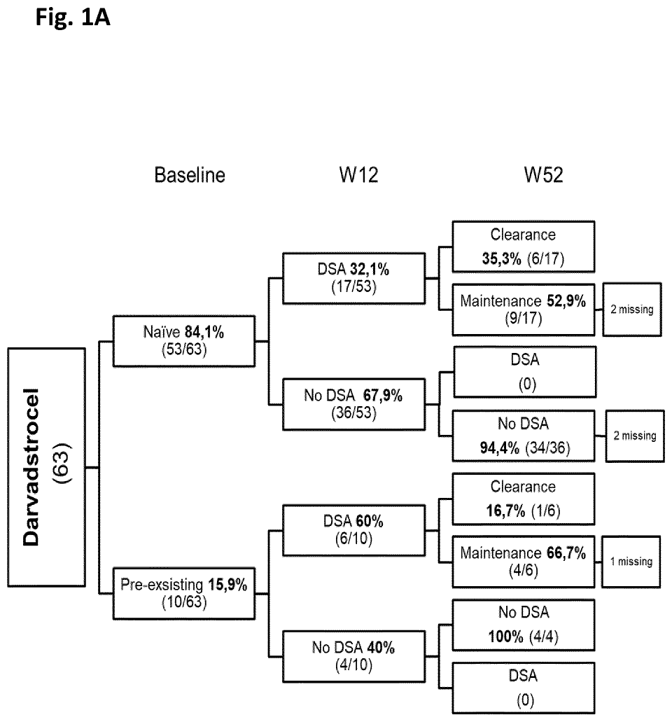

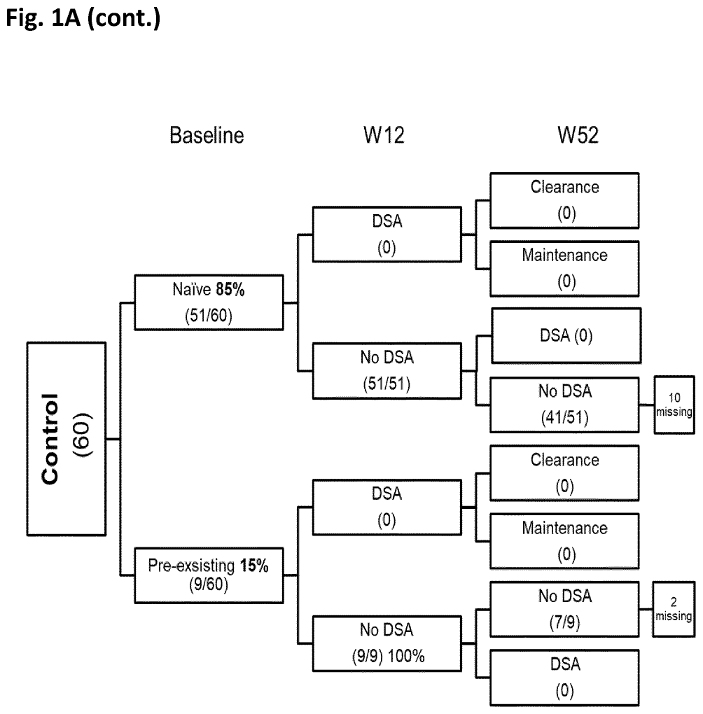

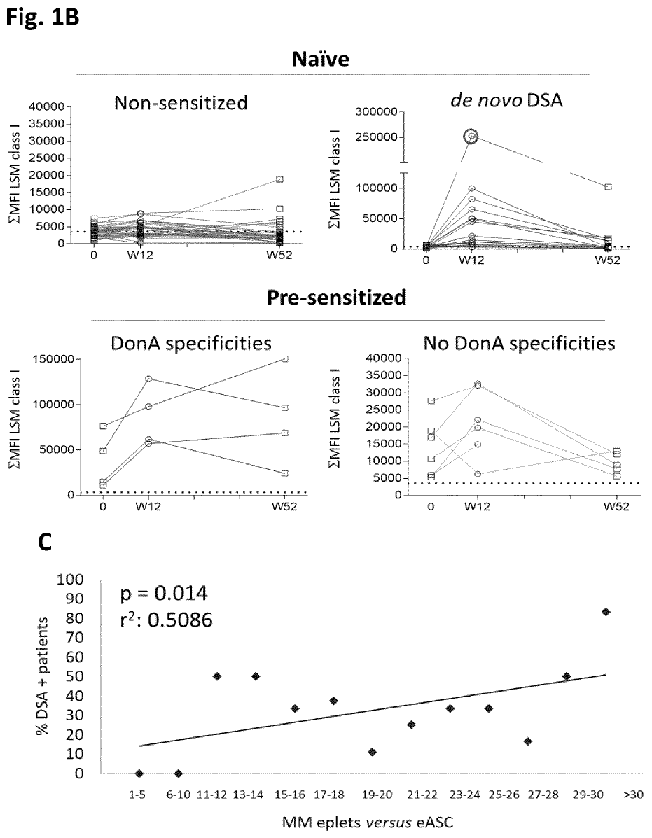

Blood samples were collected from 123 patients of the ADMIRE CD1 trial reported in Panes, J. et al. (2016), Lancet 388, 1281-1290, of them 63 ASCs and 60 placebo, at baseline and 12 weeks after treatment administration. At 52 weeks after treatment administration, 105 patients (58 ASCs and 47 placebo) provided blood samples (FIG. 1A). Analysis by solid phase assay using Luminex technology revealed that 23 patients generated DSAs 12 weeks after treatment. As expected, no patients receiving placebo generated samples with DSAs (FIG. 1A, right chart). Additionally, results indicated that 16% (10/63) of ASC patients and 15% (9/60) of placebo patients were pre-sensitized at baseline. Out of the 53 treatment-naïve patients, 17 generated ASC DSAs at W12, and six of the ten pre-sensitized patients generated ASC DSAs at W12 (FIG. 1A, left chart).

In all cases specificities of DSAs were only detected against HLA class I molecules and not against HLA class II molecules. Long-term follow-up revealed that no additional generation of DSAs was detected at W52; 30% (7/23) of patients had cleared DSAs at this time point. Interestingly, the group of treatment-naïve patients generating DSAs at W12 indicated a 35% (6/17) clearance rate, whereas pre-sensitized patients generating DSAs at W12 had a 17% (1/6) clearance rate at W52. Intriguingly, pre-sensitized patients were prone to a sustained humoral response at W52. In contrast, treatment-naïve patients generating DSA showed a trend returning towards their basal DSA level. To summarize, the above observations suggest that response to ASCs in these ADMIRE CD1 patients follows a primary immune response kinetic.

As stated in the methods (anti-HLA detection section), the level of DSA positivity for a given sample was selected using the most restrictive threshold of the single antigen results, i.e. according to categorical values (yes or no over a given cut-off). Nevertheless, the amount of antibody bound relative to the total antigen present on the purified HLA-coated beads (microspheres used in Luminex® technology) can also be quantified as the sum of MFI HLA class I LSM (least squares mean) microspheres. Time-course curves measuring plasma DSA titer throughout time (before treatment and 12-52 weeks post-treatment) were calculated; illustrating the response kinetics and determining the likelihood of reducing DSA levels (FIG. 1B). Patients were clustered in the following groups based on the presence of DSA: naïve patients that did not generate DSAs (from whose baseline levels were used for comparisons); naïve patients that generated DSAs after allo-ASC administration; pre-sensitized patients with specificities of the donor ASC administered; and pre-sensitized patients with no specificities against the donor cells used. As expected, naïve patients not generating DSAs did not exhibit HLA class I antibodies throughout the course of the study (FIG. 1B, upper left panel). In contrast, naïve patients that generated DSAs exhibited an increased antibody titer. In addition, the modification of the MFI from baseline to W12 was not similar across all patients, in fact, in certain patients the response seemed to be more intensive than others; patient 92 (Pat92) was the most allo-reactive (FIG. 1B, circle). However, it was evident that at W52 the MFI was reduced in all cases, which is in line with the antibody profile of a classic primary response, where an increase in the antibody titer reduces after the booster effect. In the group of pre-sensitized patients, which share specificities with the ASCs, a profile in-line with a secondary or memory response was found; the antibody titer was over the baseline levels at both W12 and W52 (FIG. 1B, lower left panel). These patients have the profile of a booster response that, in this case, tends to be limited in duration and intensity. Interestingly, similar kinetics were seen in the treatment-naïve patient group and in pre-sensitized patients who did not show ASCs specificities at baseline (FIG. 1B, lower right panel) indicative of a non-response profile.

A possible connection between donor-patient HLA matching grade and the probability to generate DSAs was examined. It was aimed to identify precursors of the allogeneic recognition by identifying polymorphic residues present in the ASC donor HLA type (eplets) that were absent in patients. Each patient's eplet was aligned with ASC donor HLA allele for mismatch quantification. In the present study allo-sensitization arose mainly against HLA class I; therefore, it was focused on characterizing loci A and B of HLA class I. The total number of eplets were correlated with patients' susceptibility to generate DSAs (FIG. 1C). Linear regression analysis showed no significant correlation between eplets mismatch and patient DSA generation 12 weeks post-administration.

In Vitro Binding of Anti-HLA Antibodies to ASCs

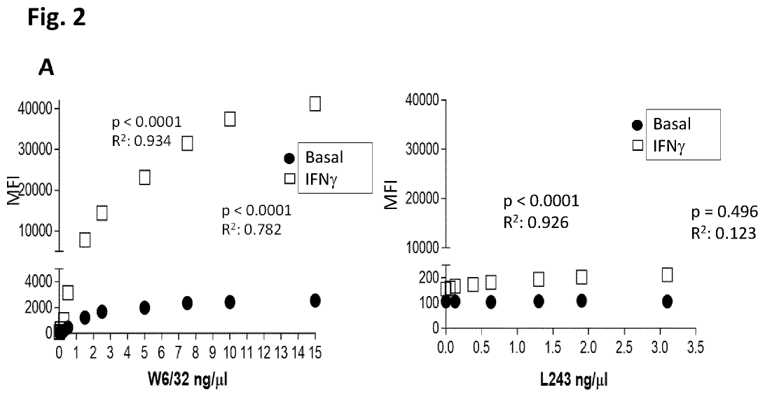

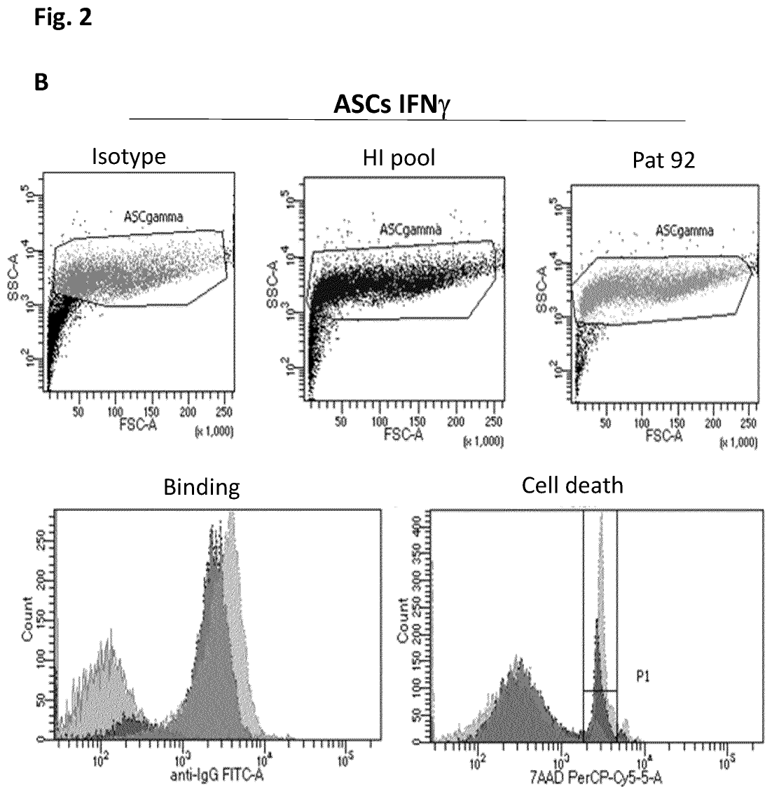

Although MSC found in different tissues share common hallmarks, including immuno-modulatory properties or identity markers, differential immunogenic responses have been reported in in vivo models. Using FACS analysis to characterize the immunogenic response of ASCs, it was intended to quantify the expression of HLA-Class I and HLA-Class II molecules on ASCs and their ability to bind to patients' anti-HLA Abs (FIG. 2A). ASCs were incubated, pre-stimulated or not with IFNγ, with increasing concentrations of fluorescence labeled HLA-Class I (weeks 6 and 32) or HLA-Class II (L243) recombinant Abs and the MFI after staining was quantified. As expected, ASCs expressed HLA-Class I at basal levels, exhibiting a strong over-expression after IFNγ stimulation (FIG. 2A). Conversely, HLA-Class II levels were negative at baseline with a modest increase following IFNγ stimulation.

The plasma sample, Pat92 (FIG. 1B, circle), was carrying the largest DSA titer post-treatment. At basal conditions, no significant increase in binding strength of DSA, or in the complement dependent toxicity assay was detected. However, when ASCs were stimulated with IFNγ, a significant upregulation of the ASCs binding strength in both the positive control (pool of hyper-immunized samples, HI pool) and Pat92 sample (FIG. 2B, lower left panel) was observed. The increase in the binding was accompanied by a high percentage of cytotoxic killing (34%) in Pat92 (FIG. 2B, light grey). This percentage was significantly higher than the percentage of killing quantified in the other 22 patients tested (ranging from 3.3-9.3%), confirming that Pat92 was the most allo-reactive sample. Interestingly, Pat92 was the one sample showing the highest level of mismatching with the administered ASCs, but it did exhibit antibody clearance at W52. Understanding the safety and immunogenic toxicity of allogeneic ASC therapy will help to evaluate the feasibility of re-treatment. Additionally, it will help determine their potential impact in pre-sensitized patients and/or in patients with exacerbated de novo DSA generation (e.g. Pat92).

Plasma DSA Binds ASCs Inducing Moderate Killing In Vitro

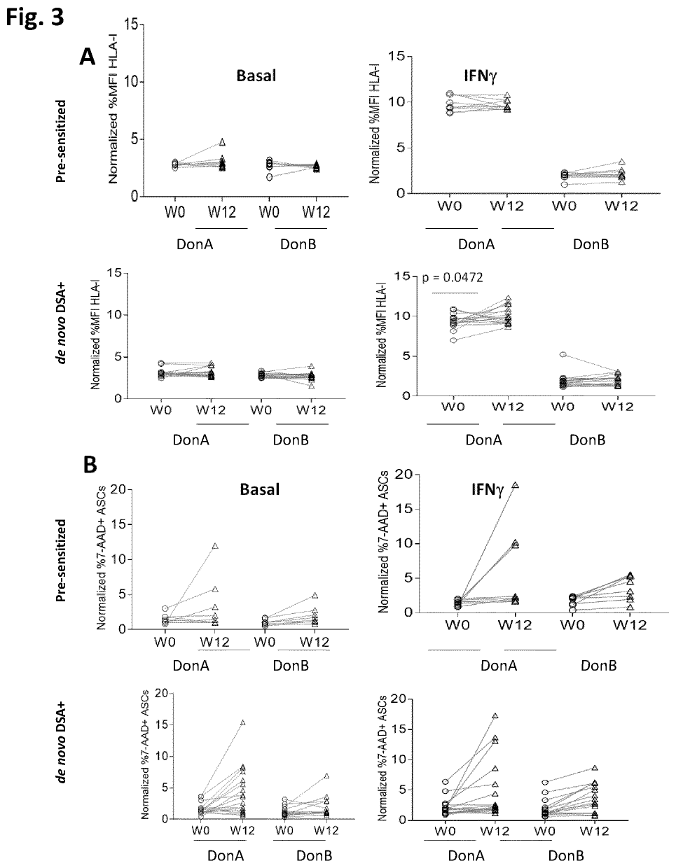

In the results above it was shown that from a cohort of 63 ADMIRE CD1 patients, 10 had pre-existing HLA-Class I Ab and 17 generated de novo DSAs. It was also demonstrated that ASCs express HLA-Class I antigen and bind to rHLA-Class I Ab; however, it is unknown whether patients' DSAs have the ability to bind and, subsequently, induce the cytotoxic killing of ASCs. To test this, it was intended to quantify the differential affinities of pre-sensitized and de novo DSA positive (DSA+) groups for binding HLA class I antigens to ASCs in vitro. In addition to the original donor, DonA (administered in the ADMIRE CD1 trial), an additional donor, DonB, was included to function as a control for DSA specificity since it was not administered to the patients (FIG. 3A). Baseline and W12 samples via FCXM and measured HLA-I binding strength to ASCs cultured in basal conditions or pre-stimulated with IFNγ (FIG. 3A, upper panels). No high-binding capacity in samples from either pre-sensitized or de novo DSA+patients in donor ASCs was observed under basal conditions. When ASCs were pre-stimulated with IFNγ, it was observed that pre-sensitized patients had high and comparable binding affinities at both week 0 (W0) and week 12 (W12) visits with DonA only (FIG. 3A, upper panels). Basal conditions and thus low HLA-I antigen expression in the membrane of ASC donors, correlated with low-binding affinity in both DonA and DonB (FIG. 3A, lower panels). Remarkably, a significant increase in binding affinity was observed in a comparison of the patient's samples derived from DonA at W12 versus baseline in de novo DSA+ samples (FIG. 3A, lower panels). The above data suggest that the concentration of HLA in basal ASCs is not sufficient to show a significant binding of DSAs in vitro. Additionally, the concentration of DSAs in most of the patients does not seem to be sufficient to show a significant binding strength, both in non-activated or activated ASCs. These results are in agreement with solid transplant flow cytometry cross-matching observations from organ transplantation studies where only patients with high DSA levels, who also share immune specificities with a donor, resulted in significant HLA-Class I binding. Next it was aimed to understand whether pre-existing HLA-I antibodies and DSA generated after allogenic administration were able to fix complement, and therefore trigger in vitro CDC. A CDC assay via flow cytometry analysis (FCtox) was optimized where 7-AAD incorporation was established as cell death readout. Plasma samples from pre-sensitized and de novo DSA+ patients (from baseline and W12) with DonA and DonB ASCs were incubated in the presence of rabbit C3 complement in either basal conditions or prior to IFNγ stimulation. In the cohort of pre-sensitized patients, W12 samples induced modest cytotoxic killing in DonB (0-5% 7-ADD+ cells) both in basal and IFNγ conditions. As seen in DonA ASCs the incubation of same samples induced higher cytotoxic killing in basal (two patients) and IFNγ (three patients) conditions (FIG. 3B). De novo DSA+ W12 samples were also able to fix complement and induce cytotoxic killing in basal conditions. As anticipated, a higher percentage of cell death was obtained only in DonA ASCs when stimulated with IFNγ (FIG. 3B). These data suggest that pre-existing HLA-Class I Abs and DSA are able to bind ASCs and induce modest CDC specifically in DonA ASCs. Interestingly, although DonB did not induce significant cytotoxic percentages, neither in pre-sensitized nor in de novo DSA+ cohorts, a trend of increased cell death in W12 versus baseline was observed. One possible explanation for that could be the existence of shared HLA polymorphic alleles between the two donors used in this study. HLA typing was performed and found that DonB shares a common allele with DonA.

DNA from DonA and DonB was purified and tested by LABType®SSO assay for HLA allele characterization. In bold, the HLA-A allele shared by DonA and DonB is shown (see Table 1).

TABLE 1

| DonA typing | DonB typing |
|---|---|
| A*02:05 A*24:02 | A*02:01 A*02:05 |
| B*15:01 B*49:01 | B*44:03 B*51:01 |
| C*03:03 C*07:01 | C*15:02 C*16:01 |
| DRB1*01:01 DRB1*11:01 | DRB1*07:01 DRB1*07:01 |
| DQB1*03:01 DQB1*05:01 | DQB1*02:02 DQB1*02:02 |
| DQA1*01:01 DQA1*05:05 | DQA1*02:01 DQA1*02:01 |

This shared HLA allele might be responsible for the trend towards increased cytotoxic levels in W12 samples in DonB.

### High Expression of mCRPs in ASCs

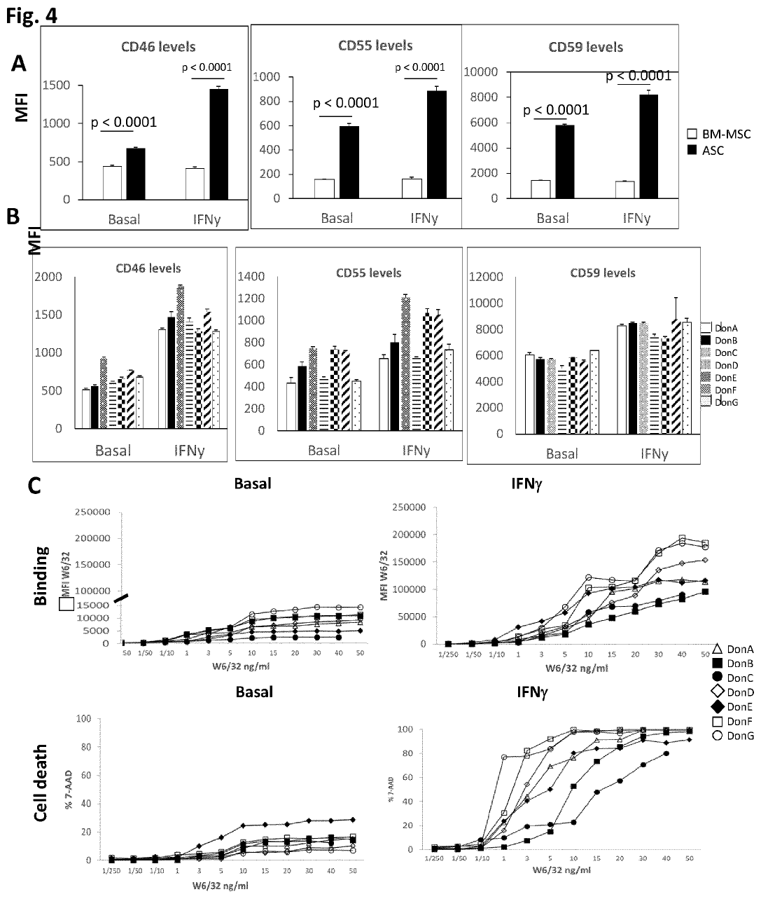

In order to understand the moderate killing of ASCs impinged by DSAs it was intended to identify complement inhibition strategies that might enable ASCs to cope and/or evade cytotoxic killing. One classical mechanism for complement signaling inhibition is the induction of mCRPs CD46, CD55 and CD59 (Gancz and Fishelson, 2009;Ricklin et al., 2010;Tegla et al., 2011). While some authors have shown that MSCs express low levels of CD46 and CD55, and high levels of CD59, others suggest that MSCs express moderate levels of all mCRPs. To address this controversy, CD46, CD55 and CD59 expression levels were analyzed in a panel of ASCs stimulated or non-stimulated with IFNγ and compared expression levels with commercial BM-MSCs (FIG. 4A). It was observed that basal levels of CD46, CD55 and CD59 were higher in ASCs compared with BM-MSCs. To repeat this physiologically relevant scenario, mCRP levels were tested in the presence of IFNγ (pro-inflammatory environment); a critical mediator of ASC immune-modulatory response. No significant modulation of mCRPs in BM-MSCs was observed, whereas ASCs appeared to induce mCRP, potently after IFNγ stimulation. CD46 induction was particularly prominent in ASCs with an approximate 2.14-fold increase compared with BM-MSCs. The above results suggest that ASCs strongly express mCRPs under basal conditions and expression is further enhanced in the presence of IFNγ. This would suggest a prominent role of mCRP in the negative regulation of CDC and hint at a cytoprotective mechanism in ASCs for coping with DSA-induced cytotoxicity. This could explain the moderate killing levels imposed by DSA.

While the expression pattern of mCRP in different ASC donors was comparable, some donors exhibited specific expression of a particular mCRP (FIG. 4B). Specifically, DonC exhibited higher CD46 and CD55 levels compared with other donors; DonE and DonF preferentially over-expressed CD55. It was further investigated whether the differential expression of mCRPs could impinge differential sensitivities to CDC. To answer this, the kinetics of HLA-I antigen expression and its binding affinities among this panel of ASC donors were investigated. In order to test this, ASC donors were subjected to increased concentrations of rHLA-Class I W6/32. Next, their binding affinity was measured via FACS (FIG. 4C). Different binding affinities among donors in basal conditions (i.e. a 12-fold difference when comparing DonC with DonG with 10 ng/mL W6/32) were noted. Following IFNγ stimulation HLA-I antigen was induced in ASC donors as indicated by increased W6/32 binding (FIG. 4C). Again, varying binding affinities among donors (6.25-fold difference at 10 ng/mL when comparing DonB with DonG) were observed. In parallel, the sensitivity of the different ASC donors to the CDC assay was tested. Under basal conditions DonE exhibited ˜25% cell death at the highest W6/32 concentrations, in the rest of the donors this was around ˜15%, except DonD and DonG which exhibited a lower percentage (FIG. 4C). As predicted, following IFNγ stimulation CDC sensitivity levels increased dramatically in all ASC donors, but to a lesser extent in DonB and DonC that remained relatively resistant to CDC-mediated cell death (FIG. 4C).

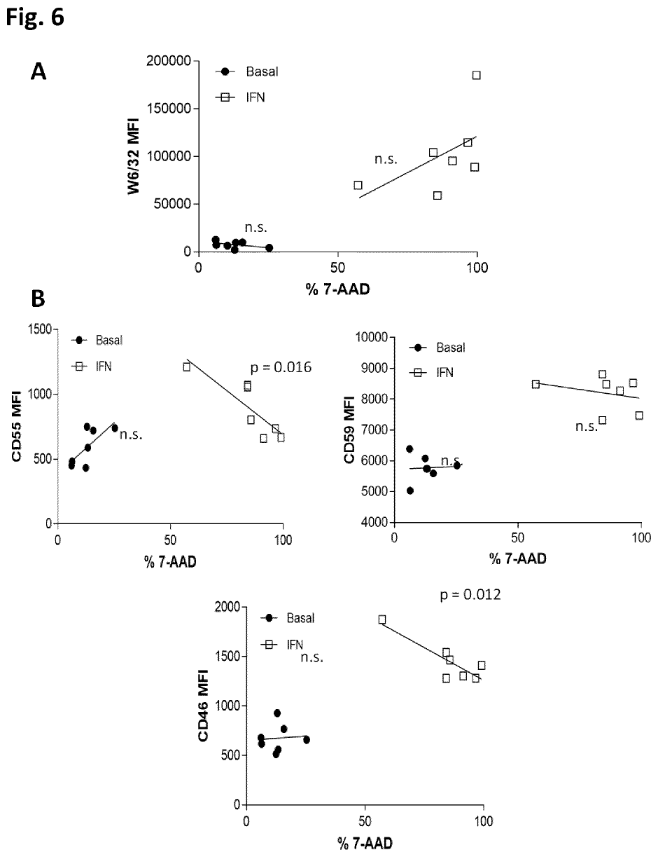

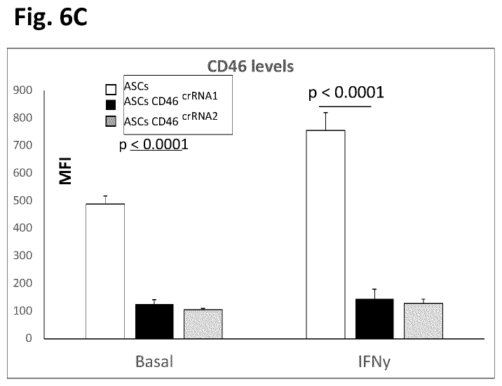

Next, it was intended to determine whether high W6/32 binding affinity correlated with enhanced CDC sensitivity. For performing this correlation analysis, a W6/32 concentration was set to 10 ng/mL as this was the Ab amount driving the transitional phase of the curves (after exponential and before plateau). No significant correlation either in basal conditions nor in the IFNγ stimulated scenario was observed (FIG. 6A). Remarkably, we identified a group of ASC donors that despite expressing relatively low W6/32 Ab MFI levels (low binding) they exhibited high sensitivity to CDC. This suggests that there is not an absolute correlation between W6/32 Ab binding and sensitivity to CDC. To determine which of the three mCRPs is the contributor to CDC inhibition of ASCs, we correlated cell death levels reached with 10 ng/mL of W6/32 with MEI expression levels at both basal and IFNγ conditions (FIG. 6B). No positive correlation was observed in basal conditions with any of the tested mCRP molecules. However, it was noticed that following IFNγ stimulation, lower CD46 and CD55 levels significantly correlated with higher cell death levels. Finally, CD46 slope significance was slightly higher compared with CD55.

### CD46 Depletion Increases CDC Sensitivity of ASCs In Vitro

The robust induction of CD46 following IFNγ stimulation compared with CD55 or CD59, and the higher significance of the cytotoxicity correlation together with the reduced inter-donor variability of CD46 versus CD55, prompted us to perform an in-depth analysis of CD46 and its potential impact in CDC sensitivity in ASCs. Using public genome browsers the top gRNA sequences to knock-down CD46 (ncbi.nlm.nih.gov/gene and crispr.mit.edu) was identified. The optimal guide RNA (gRNA)sequences was selected based on two parameters: high specificity and low off-target score. Two optimal crRNAs (crRNA 1 and crRNA2) targeting exon 3 were selected for efficacy screening. Delivery of crRNA:tracrRNA-ATT⁵⁵⁰:Cas9 complexes were examined under the microscope. It was observed that after 24 hours of lipotransfection the vast majority of ASCs had incorporated the ribonucleo-protein complexes that correlated with high Cas9-mediated double-strand break events (FIG. 6C). To check CRISPR-mediated knock-down efficacy, ASCs were cultured in the presence or absence of IFNγ and analyzed CD46 expression via FACS. The efficacy of crRNA1 was comparable to crRNA2 both under normal and IFNγ conditions, thus we selected crRNA1 for the generation of ASCs-CD46^{KO} clones (FIG. 6D).

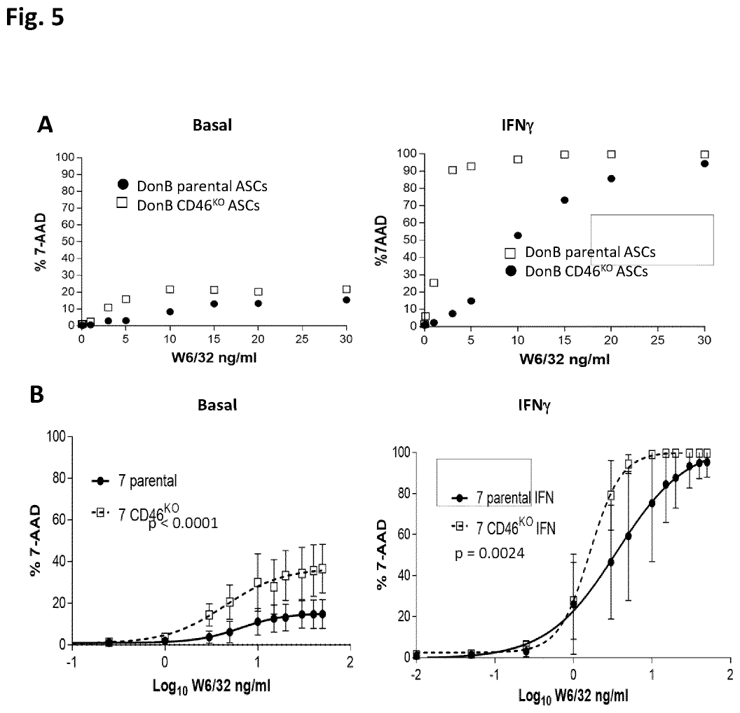

After confirming the knock-down specificity, one donor with low CDC sensitivity was selected to test whether selective depletion of CD46 could sensitize it to cytotoxic killing. DonB was selected and W6/32-mediated cytotoxic assays in basal and IFNγ conditions (FIG. 5A) was performed. CD46 knock-down induced a modest increase in cytotoxic killing under basal conditions (FIG. 5A, left graph), which was likely due to the low HLA-I binding and subsequent complement fixation. Following IFNγ stimulation a significant boost in the percentage of cytotoxic killing in parental ASCs was observed, this was further enhanced in CD46^{KO} ASCs (FIG. 5A, right graph). At a physiological dose of 10 ng/mL W6/32, ˜50% 7-AAD positive cells in parental ASCs was observed and CD46 knock-down increased the percentage of killing up to ˜95%, suggesting that CD46 plays a critical function in preventing cytotoxic killing, at least in DonB.

To test whether CD46 cytotoxic inhibitory functions are effective in other ASC donors, a cytotoxicity analysis was performed in the panel of seven donors and plotted mean curves from basal conditions (FIG. 5B, left graph) and prior to ASC IFNγ stimulation (FIG. 5B, right graph). Under both testing conditions CD46^{KO} ASC donors exhibited enhanced sensitivity to CDC compared with parental controls. A shift to the left of the half maximal effective concentration (EC₅₀) curve implies that a decrease in the concentration of the W6/32 Ab is required to induce CDC, which correlates with enhanced sensitivity to CDC. Next, to quantify the shift in the curves we calculated EC₅₀ of W6/32 Ab (FIG. 5C).

The following Table 2 shows the half-maximal effective concentration of W6/32 Ab (ng/mL) of seven parental and the corresponding CD46^{KO} ASCs in basal and IFNγ conditions. In columns 4 and 7, fold-change differences (CD46^{KO} EC₅₀/parental EC₅₀) were calculated.

| W6/32 | Basal | | | IFNγ-induced | | |
|---|---|---|---|---|---|---|
| EC₅₀ | Par- | | Fold | Par- | | Fold |
| (ng/mL) | ental | CD46^{KO} | change | ental | CD46^{KO} | change |
| DonA | 8.2 | 4.3 | 0.5 | 3.5 | 1.6 | 0.4 |
| DonB | 10.3 | 3.3 | 0.3 | 9.7 | 1.4 | 0.1 |
| DonC | 6.0 | 3.9 | 0.6 | 24.7 | 1.5 | 0.1 |
| DonD | 17.0 | 3.4 | 0.2 | 2.8 | 3.1 | 1.1 |
| DonE | 4.5 | 3.6 | 0.8 | 3.7 | 0.6 | 0.2 |
| DonF | 6.9 | 5.1 | 0.7 | 1.5 | 1.4 | 0.9 |
| DonG | 7.7 | 5.4 | 0.7 | 0.6 | 2.1 | 3.8 |

Under basal conditions, CD46^{KO} donors exhibited decreased EC₅₀ compared with parental donors, suggesting higher sensitivity to W6/32 (FIG. 5C, fourth column). A similar effect in IFNγ conditions was observed (FIG. 5C, seventh column), confirming that CD46 expression confers CDC resistance to ASCs in vitro. This data confirms that CD46 mediates CDC and that its depletion in ASCs correlates with enhanced CDC sensitivity.

1. An in vitro method for selecting a stem cell (SC) population suitable for allogeneic therapy, in particular for treatment of presensitized patients or retreatment of patients with allogeneic therapy, comprising the following steps:
a) culturing a sample of an SC population in the presence of an IFN-γ concentration capable of inducing maximal HLA-Class I expression in said SC population (test sample) and separately culturing a sample of the SC population in the absence of IFN-γ (control sample);
b) contacting the test sample and the control sample with a range of different concentrations of an HLA-Class I antibody under conditions such that the HLA-Class I antibody binds to HLA-Class I expressed in the test sample and the control sample;
c) adding complement to the test sample and the control sample such that the bound HLA-Class I antibody is saturated with complement and complement-dependent cytotoxicity (CDC) is induced;
d) determining the CDC for the range of different concentrations of the HLA-Class I antibody by measuring the cell lysis induced in the test sample and the control sample;
e) determining the concentration of HLA-Class I antibody that induces 50% of the maximal CDC (EC₅₀ value) in the test sample and the control sample; and
f1) selecting the SC population for allogeneic therapy if the ratio of the EC₅₀ value of the control sample to the EC₅₀ value of the test sample is less than 1.25, preferably less than 1.0, more preferably less than 0.5; particularly preferably less than 0.25; or
f2) selecting the SC population for allogeneic therapy if the EC₅₀ value of the test sample is at least 3.5 ng/ml, preferably at least 9 ng/ml, more preferably at least 15 ng/ml, particularly preferably at least 20 ng/ml of the HLA-Class I antibody.

2. The in vitro method of claim 1, wherein the method further comprises the steps of determining the CD46 expression level in the test sample and in the control sample; and selecting the SC population for allogeneic therapy if the ratio of CD46 expression in the test sample to the CD46 expression in the control sample is more than 2.0, preferably more than 2.5, particularly preferably more than 3.0.

3. The in vitro method of claim 1 or 2, wherein the SC population is a mesenchymal stem cell (MSC) population, preferably a human MSC population, more preferably the mesenchymal stem cell population is a bone-marrow-derived stem cell (BM-MSC) population or the mesenchymal stem cell population is an adipose tissue-derived stem cell (ASC) population, even more preferably the ASC population expresses CD29, CD73, CD90 and/or CD105.

4. The in vitro method of any one of claims 1 to 3, wherein the IFN-γ concentration capable of inducing maximal HLA-class I expression in said SC population is from about 0.5 to about 30 ng/ml, preferably from about 1 to about 15 ng/ml, more preferred from about 2 to about 4 ng/ml, preferably the IFN-γ concentration capable of inducing maximal HLA-class I expression in said SC population is 3 ng IFN-γ/ml, preferably applied over a time period of 48 hours.

5. The in vitro method of any one of claims 1 to 4, wherein the HLA-Class I antibody specifically binds to HLA-A, HLA-B and/or HLA-C, preferably the HLA-Class I antibody specifically binds to HLA-A, HLA-B and HLA-C, more preferably the HLA-Class I antibody is a murine monoclonal antibody, even more preferably the HLA-Class I antibody has essentially the same binding affinity for HLA-A as the antibody produced by the hybridoma clone w6/32 obtainable from ATCC (designation: HB-95) or ECACC (No.: 84112003), most preferred the antibody is produced by the hybridoma clone w6/32 obtainable from ATCC (designation: HB-95) or ECACC (No.: 84112003).

6. The in vitro method of any one of claims 1 to 5, wherein two or three different concentrations of the HLA-Class I antibody within the range of from about 1 to about 50 ng/ml are used, preferably four or five different concentrations of the HLA-Class I antibody within the range of from about 1 to about 50 ng/ml are used, more preferably six or seven different concentrations of the HLA-Class I antibody within the range of from about 1 to about 50 ng/ml are used, even more preferably eight or nine different concentrations of the HLA-Class I antibody within the range of from about 1 to about 50 ng/ml are used and , most preferably the range of different concentrations of the HLA-Class I antibody is 1 ng/ml, 3 ng/ml, 5 ng/ml, 10 ng/ml, 15 ng/ml, 20 ng/ml, 30 ng/ml, 40 ng/ml and 50 ng/ml.

7. The in vitro method of any one of claims 1 to 6, wherein the complement used in step (c) of claim 1 is from serum, preferably the serum has not been treated with heat, more preferably the serum is from rabbit, goat or sheep, most preferably, the serum is from rabbit.

8. The in vitro method of any one of claims 1 to 7, wherein the complement is from serum and the resulting serum concentration is from 50 to 83.33% (v/v), preferably the resulting serum concentration is from 66.66 to 83.33% (v/v).

9. The in vitro method of any one of claims 1 to 8, wherein the cell lysis is determined by measuring a chemiluminescent or fluorescent dye selective for viable or lysed cells or a radioactive agent released from lysed cells.

10. An SC population suitable for allogeneic therapy, in particular for the treatment of presensitized patients or retreatment with allogeneic therapy, having any of the following properties:
i) a ratio of the EC₅₀ value of the control sample to the EC₅₀ value of the test sample of less than 1.25, preferably less than 1.0, more preferably less than 0.5 and particularly preferably less than 0.25, wherein the EC₅₀ value is determined as set forth iri claim 1;
ii) an EC₅₀ value of the test sample of at least 3.5 ng/ml HLA-Class I antibody, preferably at least 9 ng/ml, more preferably at least 15 ng/ml, particularly preferably at least 20 ng/ml HLA-Class I antibody, wherein the EC₅₀ value is determined as set forth in claim 1; and/or
iii) a ratio of CD46 expression in the test sample to the CD46 expression in the control sample is more than 2.0, preferably more than 2.5, particularly preferably more than 3.0, wherein the CD46 expression is determined as set forth in claim 2.

11. The SC population according to claim 10, wherein the SC population is selected by the method of any one of claims 1 to 9, preferably the SC population is a mesenchymal stem cell (MSC) population, more preferably the SC population is a BM-MSC population or an ASC population, even more preferably the SC population is a human BM-MSC population or a human ASC population.

12. A pharmaceutical composition comprising the SC population according to claim 10 or 11 and optionally a pharmaceutically acceptable carrier.

13. A method for preparing a pharmaceutical composition comprising the steps:
a) performing the method of any one of claims 1 to 9;
b) formulating the selected SC population with at least one pharmaceutically acceptable carrier.

14. The SC population according to claim 10 or 11 for use in allogeneic stem cell therapy in a patient in need thereof, preferably in a presensitized patient or a patient undergoing retreatment, preferably the patient in need suffers from a disease selected from fistulas, leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease, more preferably the disease is a fistula, even more preferably the disease is a complex perianal fistula.

15. An allogeneic stem cell therapy method comprising administering the SC population according to claim 10 or 11 to a patient in need thereof, preferably in a presensitized patient or a patient undergoing retreatment, preferably the patient in need suffers from a disease selected from leukemia, lymphoma, neurodegenerative diseases, brain and spinal cord injury, heart diseases, blindness and vision impairment, pancreatic beta cell loss of function, cartilage repair, osteoarthritis, musculoskeletal diseases, wounds, infertility, autoimmune diseases and inflammatory diseases such as inflammatory bowel disease, more preferably the disease is a fistula, even more preferably the disease is a complex perianal fistula.
